(19) United States
(12) Patent Application Publication
(10) Pub. No.: US 2014/0044644 A1
(43) Pub. Date: Feb. 13, 2014

(54) ANKYRIN G AND MODULATORS THEREOF FOR THE TREATMENT OF NEURODEGENERATIVE DISORDERS

(71) Applicants: Antonella Santuccione Chadha, Thalwil (CH); Mario Merlini, San Francisco, CA (US); Roger Nitsch, Zumikon (CH); Jan Grimm, Dubendorf (CH); Christoph Hock, Erlenbach (CH)

(72) Inventors: Antonella Santuccione Chadha, Thalwil (CH); Mario Merlini, San Francisco, CA (US); Roger Nitsch, Zumikon (CH); Jan Grimm, Dubendorf (CH); Christoph Hock, Erlenbach (CH)

(73) Assignee: UNIVERSITY OF ZURICH, Zurich (CH)

(21) Appl. No.: 14/000,454

(22) PCT Filed: Feb. 21, 2012
(86) PCT No.: PCT/EP12/52907

§ 371 Date: Oct. 30, 2013

Related U.S. Application Data
(60) Provisional application No. 61/445,447, filed on Feb. 22, 2011.

(30) Foreign Application Priority Data
Feb. 21, 2011 (EP) 11001406.5

(51) Int. Cl. A61K 39/395; G01N 33/68; A61K 31/7088; A61K 49/00; A61K 38/17; A61K 31/713

(52) U.S. Cl. CPC A61K 39/3955; A61K 38/1709; A61K 31/713; A61K 31/7088; A61K 49/0004; G01N 33/6896
USPC 424/9.1; 514/1.2; 424/184.1; 514/44 R; 514/44 A; 435/7.1; 435/7.92; 424/158.1

(57) ABSTRACT

The present invention generally relates to the technical field of medicine, in particular to the field of neurodegenerative, neurological and protein misfolding disorders such as amyloidosis. By establishing a role of ankG in APP processing the method of the present invention provides a new insight into the role of ankG in AD pathology and provides ankG as a target, drug, diagnostic agent and particularly as a vaccine in the treatment and diagnosis of the pathogenesis of Alzheimer's disease (AD).

### FIELD OF THE INVENTION

The present invention generally relates to the technical field of medicine, in particular to the field of neurodegenerative, neurological and protein misfolding disorders such as amyloidosis. More specifically, the invention relates to a surprising role of ankyrin G (ankG) in β-amyloid (Aβ) clearance and in amyloid precursor protein (APP) metabolism.

### BACKGROUND OF THE INVENTION

The neuronal protein ankyrin G (ankG/ANKG) is a member of the highly conserved family of ankyrin proteins which are involved in anchoring and transporting membrane and cytoplasmic proteins to the actin-spectrin cytoskeleton (1). Ankyrin G (also known as ankyrin 3, ANK3, and node of Ranvier (ankyrinG)) is part of the actin-spectrin cytoskeleton of neurons, forming an intracellular scaffolding protein which directs and anchors proteins to specialised membrane domains such as the axon initial segment (AIS) of hippocampal neurons (2), indicating a role of ankG in intracellular transport. For example, neural cell adhesion molecules including L1CAMs are localised to the neuronal cell membrane by ankG (3, 4). Targeted disruption of ankG expression in the mouse cerebellum resulted in progressive ataxia and abolished localisation of voltage-gated Na⁺ channels and NrCAMs molecules to the AIS and also progressive Purkinje neuron degeneration is known to occur (4). Recently, ankG was found to be of essential importance in coordinating the trafficking and the ordered distribution of molecules which are present at the AIS of hippocampal neurons (5). In addition, different classes of transmembrane channels require ankG to be properly located. For instance, ankG is required for transport of cyclic nucleotide-gated channels to the plasma membrane of rod photoreceptor sensory cilia outer segments (6). The regulated targeting and concentration of these proteins not only help the adhesion between cells but also cell signalling. Furthermore, ankG expression determines the concentration and accumulation of proteins targeted to the AIS which is exemplified by the fact that any AIS protein not bound to ankG and hence the spectrin cytoskeleton are being endocytosed and are not expressed at the cell membrane (7). This ability of ankG to regulate protein densities at the cell membrane is of utmost importance for proper neuronal cell functioning. It can thus be understood that a (pathological) over-expression of ankG could lead to accumulation of membrane proteins which insertion is mediated and regulated by ankG. This is an intriguing hypothesis, especially when looked at from an aberrant protein processing and transporting point of view in neurodegenerative disorders including Alzheimer's disease (AD). Interestingly, scaffolding proteins like ankG have recently been identified as important key players in regulating signalling pathways which control the immune response (8). Besides its capability of transporting and anchoring transmembrane proteins, ankG is involved in the formation of specialised membrane micro-domains (9). This puts ankG in a highly dynamic and essential position in the organisation of the functionality of the neuronal membrane. Transport of ankG to the AIS is thought to be mediated by the regulation of the activity of nuclear factor κB (NFκB) and not by the membrane proteins it binds to (10). The phosphorylated, that is, inactive form of IκB-alpha (IκBα), one of the inhibitors of NFκB, has been discovered to be enriched at the AIS and its activation results in the failure of ankG to be targeted to the AIS, leading to accumulation of ankG in the neuronal cytosol (11). Such disturbances in the NFκB pathway are known to occur in neurodegenerative disorders including Alzheimer's disease (AD) (12). Cytoskeletal disturbances, impaired intraneuronal transport and changes in neuronal membrane dynamics are known factors in AD (13). Interestingly, a possible role for the cytoskeletal protein ankG in AD pathology was suggested by genetic studies showing that the gene encoding for ankG is located on chromosome 10 within a genetic interval linked to late-onset AD (14). Additionally, ankG mRNA expression was found to be about two-fold higher in AD patients (15). As a further potential link to neurological disorders, the ANKG gene locus has been found as one of significant bipolar disorder loci tested in schizophrenia (Ferreira et al., Nat. Genet. 40 (2008), 1056-1058).

Despite numerous scientific efforts in the last years, no means to cure AD has been found yet but drug treatment methods that delay or ameliorate the symptoms in the early stages only. Since an increasing number of factors has been identified as a possible cause for AD, it is likely that a combination of factors leads to the development of AD in an individual.

As mentioned above, ankG may be one of these responsible factors and have a role in the development of neurodegenerative disorders including Alzheimer's disease. These findings provide thus a basis for a requirement for drugs targeting ankG and its interaction partners as a possible new means to diagnose, prevent, delay and ameliorate the course or to cure said neurodegenerative disorders. These requirements are solved by the embodiments characterized in the claims and described further below.

### SUMMARY OF THE INVENTION

Generally, the present invention relates to neuronal protein ankyrin G (ankG/ANKG) both as a drug and diagnostic means as well as drug and diagnostic target especially in the field of neurological degenerative disorders.

The present invention is based inter alia on the surprising finding of ankG to be both abnormally re-distributed and overexpressed within the neuronal soma and to be present extracellularly in AD brain, localised within β-amyloid plaques. Without intending to be bound by theory, it is hypothesized that this pathological extracellular presence of ankG could lead to an immune response against ankG in AD. Indeed, anti-ankG antibodies were found in AD sera. Surprisingly, endogenously-produced ankG antibodies following active vaccination of APP-transgenic mice with ankG significantly reduced β-amyloid plaque pathology. In addition, it was found that anti-ankG antibodies were protective against Aβ-induced dendritic spine loss. Obtained data establishes also a role for ankG in APP processing and AD pathology. Altogether, the findings of the present invention support a neuroprotective effect of the endogenous anti-ankG response in AD.

Hence, by establishing a role of ankG in APP processing the present invention provides a new insight into the role of ankG in AD pathology and establishes ankG as a target, drug, diagnostic agent and particularly as a vaccine in the treatment and diagnosis of the pathogenesis of Alzheimer's disease (AD).

### BRIEF DESCRIPTIONS OF THE DRAWINGS

FIG. 1: AnkG is present in β-amyloid plaques
(a) AnkG was present within β-amyloid plaques in the hippocampi of AD patients. Immunohistochemical analysis of paraffin sections from the hippocampi of AD patients was performed using anti-mouse antibody recognizing ankG (red) and anti-human antibody recognizing Aβ (brown). Note the expression of ankG in neurons in both healthy control subjects (HCS) and AD samples (arrow). AnkG-positive structures resembling dystrophic neurites were found in plaques (arrowhead). Scale bar=50 μm. (b) AnkG was present in neuritic plaques in brains from arcAbeta mice. Immunohistochemical staining on 6 and 12 months old arcAbeta mice brains was performed using monoclonal antibodies recognizing ankG (red) and polyclonal antibodies recognizing the C-terminal portion of APP (green). Note the presence of both ankG and APP along the axonal initial segment (AIS) of cortical neurons in the young mouse cortex (arrowhead). In the 12 months old arcAbeta mouse cortex ankG-positive structures were present in the β-amyloid plaques (arrowhead) as seen in the human β-amyloid plaques (d). AnkG immunoreactivity was also observed within dystrophic neurons together with APP. Scale bar=20 μm. (c) Bar graph quantifying ankG immunoreactivity observed in the frontal cortex of AD brains as compared to HCS using tissue micro array analysis. Higher protein levels of ankG were seen in AD versus HCS frontal cortex. Student's t test, p=0.02 (n=12).

FIG. 2: Expression of ankG is deregulated in AD brains and in arcAbeta mouse brains
(a) WB analysis of SDS soluble and insoluble fractions isolated from AD and HCS hippocampi for ankG, Aβ, tau and APP. AnkG redistributed in the same fraction as Aβ in AD human hippocampus. Note the increased presence of ankG, Aβ and tau in the SDS insoluble fraction from AD as compared to HCS. GAPDH staining indicates that the same amount of total protein was loaded in the AD and HCS lanes for each fraction. (b) WB analysis of SDS insoluble fractions from AD versus HCS frontal cortex. Note the increased expression of ankG in the SDS insoluble fraction of the AD affected samples compared to HCS. GAPDH was used as a loading control. (c) Immunofluorescence localization of ankG in hippocampal sections of arcAb eta mice and their non-transgenic (NTG) littermates at 24 months of age. Note the redistribution of ankG in the arcAbeta hippocampus when compared to their non-transgenic littermates. A magnified view of the ankG immunofluorescence is presented in the third row to depict the distribution of ankG in the CA3 region of the hippocampus. The hippocampal distribution of neurofilament (nf-200) between arcAbeta mice and their non-transgenic littermates was similar. Scale bar=300 μm. (d) WB analysis of exosomal fractions obtained from HEK 293 cells shows the presence of ankG in exosomes together with the exosomal marker alix. Note the total absence of calnexin from exosomal fraction showing the purity of the preparation.

FIG. 3: AnkG can behave as an antigen triggering the production of specific antibodies in AD patients
(a) Representative WBs shown evaluating serum immunoreactivity against ankG. The immunoreactive band (≈190 kDa) against ankG shown in AD samples was absent in the HCS. (b) Bar graph depicting serum immunoreactivity against ankG evaluated by WBs. Bar graph shows that 8 out of 14 sera samples from AD patients and only 2 out of the 14 sera samples from HCS tested similarly were immunopositive for ankG. Chi-square test, p=0.018. (c) Analysis of serum IgG immunoreactivity in AD patients by protein expression arrays. Incubation of the protein expression array with sera from a representative AD patient. A 3 cm×3 cm section of the 24 cm×24 cm array is shown. IgG immunoreactivity of the AD sera to the expression clone for ankG (spotted in duplicate) is indicated by arrows.

FIG. 4: AnkG immunization of arcAbeta mice results in the production of antibodies against ankG and induces microglia-mediated clearance of β-amyloid
(a) A representative β-amyloid plaque stained with antibodies recognizing APP/Aβ (blue), ankG (red) and ionized calcium binding adaptor molecule 1 (Iba-1/green) is shown for immunized and non-immunized arcAbeta mice. Iba-1 is a specific marker of macrophages/microglial cells in the brain (Ito et al., Brain Res. Mol. Brain. Res. 57 (1998), 1-9; Thomas W E, Brain Res Brain Res Rev. 17 (1992), 61-74). Note the reduction in plaque size in ankG-immunized as compared to control-immunized arcAbeta mice. An overlapping staining was observed for APP/Aβ and ankG and APP/Aβ, ankG and Iba-1. Note the higher expression/number of Iba-1 immunoreactive cells within the plaque after ankG immunization. Scale bar=20 μm.
(b) Quantitative image analysis of β-amyloid plaques in the cortex of arcAbeta mice immunized with ankG as compared to control-immunized mice. The diameter (p=0.026) of the β-amyloid plaques and the plaque load (p=0.055) were significantly reduced in the cortex of ankG-immunized arcAbeta mice. Mean values±SEM (Student's t-test, n=3). Immunization experiments were repeated three times with 3 mice in each group for each experiment. (c) Representative WBs showing the levels of Aβ, nf-200 and GAPDH in the SDS insoluble brain fractions from ankG-immunized and control-immunized arcAbeta mice and their non-transgenic littermates. Aβ levels were decreased whereas neurofilament and GAPDH were not affected upon immunization of arcAbeta mice with ankG. Bar graphs show densitometric quantification of the WBs for Aβ levels in ankG and control-immunized arcAbeta mice. Aβ intensities were normalized to GAPDH (n=3, Mann-Whitney test, p=0.005).

FIG. 5: Monoclonal antibodies against ankG lower Aβ levels and Aβ-induced spine loss in ex vivo hippocampal slice cultures from arcAbeta mice
(a) Quantitative bar graphs representing mean values of the amount of Aβ40 and Aβ42 peptide in medium of arcAbeta hippocampal slice cultures after treatment with a monoclonal antibody against ankG (mAbA) for 1 week. ELISA analysis showed a decrease in Aβ40 and Aβ42 peptides as compared to control immunization (n=6, Student's t test, p=0.0002 for Aβ42, p=0.0003 for Aβ40). (b) Representative high-resolution confocal images of dendritic segments from CA3 apical dendrites of EGFP-expressing neurons from hippocampal slice cultures of arcAbeta mice and their non-transgenic littermates. (Stratum radiatum, scale bar: 5 μm). Spine density is strongly reduced in arcAbeta neurons. Note the protective effect of the anti-ankG antibody (mAbA) as compared to untreated arcAbeta cultures. Anti-ankG antibody treatment does not affect spine density in non-transgenic controls. (c) Quantification of spine density in EGFP-expressing neurons from hippocampal slice cultures of arcAbeta mice and their non-transgenic littermates. Spines were analyzed in CA1 and CA3 apical dendrites and the results were pooled. Values are shown as average (n=10, Student's t test, p=0.00001).

FIG. 6: AnkG interacts directly with APP in the brain
(a) AnkG and APP immunoprecipitates from human frontal cortex were immunoblotted with specific antibodies against ankG, APP and ankB. Immunoprecipitates with non-immune IgG served as a negative control. Note that APP co-immunoprecipitated with ankG and vice versa. Human brain homogenate (C) was used as positive control. (b) AnkG immunoprecipitates were obtained from arcAbeta (TG), non-transgenic (NTG) littermates and APP deficient (−/−) mouse brains. Note that APP, but not ankB, co-immunoprecipitated with ankG. Mouse brain homogenate (C) was used as positive control. (c) Primary rat hippocampal cultures were stained with antibodies against the C-terminal portion of APP (green) and with antibodies against ankG (red). Note the presence of both ankG and APP in the AIS. Scale bar=10 μm (upper panels) and Scale bar=30 μm (lower panels). (d) ELISA binding curve shows the direct interaction of purified ankG to APP at its intracellular domain (AICD50) coated on ELISA plates (5 μg/ml). AnkG binding to BSA-coated wells (2 mg/ml) served as negative control. Mean values (OD₄₅₀)±SEM are shown (n=6). (e) AnkG and APP immunoprecipitates from human AD hippocampus were immunoblotted with specific antibodies against APP and Caspr and L1. Immunoprecipitates with non-immune IgG served as a negative control. As already shown in (a) APP co-immunoprecipitated with ankG. Note the absence of Caspr and L1 from the immunoprecipitates. AD hippocampus homogenate (+) was used as positive control.

FIG. 7: Silencing of ankG results in altered trafficking of APP
(a) Surface biotinylation to analyze the effects of ankG siRNA silencing on the trafficking of APP to the cell surface in SHY-5Y neuroblastoma cells. WB analysis showed a reduction in cell surface biotinylated APP after ankG silencing as compared to non-silenced or ankB-silenced cells. The presence of the cell adhesion molecule caspr at the cell surface was not affected by silencing (n=3). (b) Immunocytochemistry of APP-citrine overexpressing HEK293 cells showed an intracellular accumulation of citrine-APP within the cells and almost a lack of APP at the cell membrane after silencing of ankG. Silencing of ankB did not affect surface localisation of APP although some intracellular accumulation could be seen. Scale bar=20 μm. (c) WB analysis of cell lysates of ankG silenced HEK293. Reduced amounts of the α-CTF were detected in lysates from cells treated with ankG siRNA as compared to cells treated with ankB siRNA and non-silenced cells. Cells were pre-treated with the γ-secretase inhibitor DAPT to obtain detectable levels of α-CTF. Actin was used as a loading control. (Student's t test, p=0.009, n=6). (d) Quantitative bar graphs showing the Aβ40 reduction as observed by ELISA in the medium of HeLa cells expressing the Swedish APP mutation after ankG silencing as compared to control silenced cells (Student's t test, p=0.004, n=9).

FIG. 8: (a) Increase in antibody titres against ankG in sera from arcAbeta mice after monthly subcutaneous immunizations with recombinant ankG protein as determined by ELISA, and plotted at different time points. Note that high serum titres of anti-ankG antibodies were detectable after the third month of immunization. (b) Mouse antibodies after ankG immunization were found within β-amyloid plaques. Scale bar=20 μm. (c) Quantitative bar graphs representing mean values of the amount of Aβ40 and Aβ42 peptide in the SDS insoluble fractions. ELISA analysis showed a decrease in Aβ40 and Aβ42 peptides in arcAbeta mice after ankG immunization as compared to control immunization (n=3, Mann-Whitney test, p=0.03 for Aβ42, p=0.04 for Aβ40). (d) ELISA assays to quantify the amount of antibodies against Aβ40 and Aβ42 peptides in arcAbeta mice after ankG immunization as compared to arcAbeta control-immunized. Note that there was no increase in anti-Aβ40 and Aβ42 antibodies after ankG immunization. (e, f) Quantitative bar graphs representing mean values of the amount of Aβ42 peptide in the SDS soluble fractions. ELISA analysis showed an increase in Aβ42 (e) (Student's t test, p=0.02, n=24) but not in Aβ40 (f) (Student's t test, p>0.05, n=24). (g) Quantitative bar graphs representing mean values of the amount of Aβ42 peptide in the formic acid extracted SDS insoluble fractions from APPSwe mice. ELISA analysis showed a decrease in Aβ42 peptides after ankG immunization as compared to controls (n=4, Student's t test, p=0.01). (h) Quantitative bar graphs representing mean values of the amount of Aβ40 peptide in the formic acid extracted SDS insoluble fractions from APPSwe mice. ELISA analysis showed no differences in Aβ40 peptides after ankG immunization as compared to controls (n=4, Student's t test, p=0.8). (i) Quantitative bar graphs representing mean values of the amount of Aβ42 peptide in the sera of APPSwe mice immunized with ankG. ELISA analysis showed an increase in Aβ42 peptides after ankG immunization as compared to controls (n=4, Student's t test, p=0.04).

FIG. 9: (a) Quantification of Iba1 mean intensity per plaque area shows an increased staining within plaques of arcAbeta mice after ankG immunization as compared to control-immunized arcAbeta mice. (b) Quantitative bar graphs representing mean values of the amount of Aβ42 peptide in the formic acid extracted SDS insoluble fractions from APPSwe mice. ELISA analysis showed a decrease in Aβ42 peptides after ankG immunization as compared to controls (n=4, Student's t test, p=0.007). (c) Quantitative bar graphs representing mean values of the amount of Aβ42 peptide in the sera of APPSwe mice immunized with ankG. ELISA analysis showed an increase in Aβ42 peptides after ankG immunization as compared to controls (n=4, Student's t test, p=0.009). (d) WB showing a strong immunoreactivity for recombinant ankG of monoclonal antibodies against ankG obtained after ankG active immunization of arcAbeta mice. Note that there is no cross-reactivity with the high homologous recombinant protein ankB.

FIG. 10: (a) ELISA binding curves indicate no direct binding between ankG and fibrillar Aβ40 or Aβ42. AnkG and Aβ42 recognized by specific antibodies served as positive controls for the functionality of the assay. Mean values (OD₄₅₀)±SEM (n=3) are shown. (b) Cell lysates were immunoblotted and analyzed for protein levels of APP, ankG, ankB and GAPDH after being treated with γ secretase inhibitor and ankG siRNA, ankB siRNA or silenced controls. Decreased levels of ankG and ankB corresponded to the ankG and ankB siRNA treatment. Full length APP was not decreased after silencing of ankG or ankB as compared to the silenced controls. GAPDH was used as a loading control (n=6). (c) Cell lysates of overexpressing Swedish APP HEK cells were immunoblotted and analyzed for protein levels of Aβ, APP and ankG after ankG silencing or silenced controls. Decreased levels of Aβ were observed after ankG silencing. Full length APP was not decreased after silencing of ankG (n=3). (d) Histogram representing the α-CTF reduction after ankG silencing as compared to cells silenced for ankB and non-silenced cells. (e) WB analysis of cell lysates of SH-SY5Y ankG silenced cells. Reduced amounts of the α-CTF were detected in lysates from cells treated with ankG siRNA as compared to cells treated with ankB siRNA. Cells were pre-treated with the γ-secretase inhibitor DAPT to obtain detectable levels of α-CTF. Actin was used as a loading control. (Student's t test, p=0.02, n=6). (f) Histogram representing the APP reduction in SDS soluble fractions after ankG immunization in arcAbeta mice. (g) SDS soluble fraction of arcAbeta immunized mice were immunoblotted and analysed for proteins levels of APP. Note the reduced presence of APP after immunization as compared to controls (Student's t test, p=0.03, n=6).

FIG. 11: AnkG-immunization is not neurotoxic and does not interfere with known physiological functions of ankG in neurotransmission
(a) Four groups of littermate mice, including non transgenic (n=23), arcAbeta (n=23), non transgenic immunized (n=23) and arcAbeta-immunized (n=25) mice, were subjected to Y-Maze. Mice were individually placed into a radial symmetric-maze and the total number of arm entries (y axis) was estimated for each group (x axis). Transgenic mice demonstrated significantly higher numbers of arm entries as compared to non-transgenic mice, as already described (17). No significant difference was found between wildtype and wildtype-immunized and transgenic and transgenic immunized. Data are represented as group means±s.e.m. All post hoc statistical comparisons are versus non transgenic mice, *p=0.05, **p=0.001. (b) Percentage alternation between Y-maze arms (y axis) is represented for each genotype (x axis). No significant difference was found between non transgenic and non transgenic-immunized and transgenic and transgenic immunized (Anova test, P>0.05). Data are represented as group means s.e.m. All post hoc statistical comparisons are versus non transgenic mice, *p=0.05, **p=0.04.

### DEFINITIONS

Unless otherwise stated, a term as used herein is given the definition as provided in the Oxford Dictionary of Biochemistry and Molecular Biology, Oxford University Press, 1997, revised 2000 and reprinted 2003, ISBN 0 19 850673 2.

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

The term “peptide” is understood to include the terms “polypeptide” and “protein” (which, at times, may be used interchangeably herein) within its meaning. Similarly, fragments of proteins and polypeptides are also contemplated and may be referred to herein as “peptides”. Nevertheless, the term “peptide” preferably denotes an amino acid polymer including at least 5 contiguous amino acids, preferably at least 10 contiguous amino acids, more preferably at least 15 contiguous amino acids, still more preferably at least 20 contiguous amino acids, and particularly preferred at least 25 contiguous amino acids. In addition, the peptide in accordance with present invention typically has no more than 100 contiguous amino acids, preferably less than 80 contiguous amino acids and more preferably less than 50 contiguous amino acids.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Nevertheless, the term “polypeptide” preferably denotes an amino acid polymer including at least 100 amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

“Recombinant peptides, polypeptides or proteins” refer to peptides, polypeptides or proteins produced by recombinant DNA techniques, i.e. produced from cells, microbial or mammalian, transformed by an exogenous recombinant DNA expression construct encoding the fusion protein including the desired peptide. Proteins or peptides expressed in most bacterial cultures will typically be free of glycan. Proteins or polypeptides expressed in yeast may have a glycosylation pattern different from that expressed in mammalian cells.

Also included as polypeptides of the present invention are fragments, derivatives, analogs or variants of the foregoing polypeptides, and any combination thereof. The terms “fragment,” “variant,” “derivative” and “analog” include peptides and polypeptides having an amino acid sequence sufficiently similar to the amino acid sequence of the natural peptide. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the preferred peptides of the present invention, in particular to ankG, APP or fragments, variants, derivatives or analogs of either of them, in particular wherein said fragment derivative or analog is derived from the intracytoplasmatic domain of APP. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

The terms “fragment,” “variant,” “derivative” and “analog” when referring to antibodies or antibody polypeptides of the present invention include any polypeptides which retain at least some of the antigen-binding properties of the corresponding native binding molecule, antibody, or polypeptide. Fragments of polypeptides of the present invention include proteolytic fragments, as well as deletion fragments, in addition to specific antibody fragments discussed elsewhere herein. Variants of antibodies and antibody polypeptides of the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of ankG specific binding molecules, e.g., antibodies and antibody polypeptides of the present invention, are polypeptides which have been altered so as to exhibit additional features not found on the native polypeptide. Examples include fusion proteins. Variant polypeptides may also be referred to herein as “polypeptide analogs”. As used herein a “derivative” of a binding molecule or fragment thereof, an antibody, or an antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

“Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a binding molecule, an antibody, or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA), small hairpin RNA (shRNA), small interfering RNA (siRNA) or any other RNA product.

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full-length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., small interfering RNA (siRNA), a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Unless stated otherwise, the terms “disorder” and “disease” are used interchangeably herein.

A “binding molecule” as used in the context of the present invention relates primarily to antibodies, and fragments thereof, but may also refer to other non-antibody molecules that bind to ankG including but not limited to hormones, receptors, ligands, major histocompatibility complex (MHC) molecules, chaperones such as heat shock proteins (HSPs) as well as cell-cell adhesion molecules such as members of the cadherin, intergrin, C-type lectin and immunoglobulin (Ig) superfamilies. Thus, for the sake of clarity only and without restricting the scope of the present invention most of the following embodiments are discussed with respect to antibodies and antibody-like molecules which represent the preferred binding molecules for the development of therapeutic and diagnostic agents.

The terms “antibody” and “immunoglobulin” are used interchangeably herein. An antibody or immunoglobulin is an ankG-binding molecule which comprises at least the variable domain of a heavy chain, and normally comprises at least the variable domains of a heavy chain and a light chain. Basic immunoglobulin structures in vertebrate systems are relatively well understood; see, e.g., Harlow and Lane, Antibodies: A Laboratory Manual, (Cold Spring Harbor Laboratory Press, 2nd ed. 1988).

Any antibody or immunoglobulin fragment which contains sufficient structure to specifically bind to ankG is denoted herein interchangeably as a “binding fragment” or an “immunospecific fragment.”

Antibodies or antigen-binding fragments, immunospecific fragments, variants, or derivatives thereof of the invention include, but are not limited to, polyclonal, monoclonal, multispecific, human, humanized, primatized, murinized or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to antibodies disclosed herein). ScFv molecules are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibodies or immunospecific fragments thereof of the present invention may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

In a particularly preferred embodiment of the present invention, the antibodies are naturally occurring human monoclonal anti-ankG antibodies or binding fragments, derivatives and variants thereof cloned from human subjects. Identification of ankG-specific B-cells and molecular cloning of anti-ankG antibodies displaying specificity of interest as well as their recombinant expression and functional characterization can be generally performed as described in the Examples and Supplementary Methods section of international application PCT/EP2008/000053 published as WO2008/081008, the disclosure content of which is incorporated herein by reference in its entirety. A new method for identification of ankG-specific B-cells and molecular cloning of ankG antibodies displaying specificity of interest as well as their recombinant expression and functional characterization is provided within this application. As described above in one embodiment of the present invention cultures of single or oligoclonal B-cells are cultured and the supernatant of the culture, which contains antibodies produced by said B-cells is screened for presence and affinity of new anti-ankG antibodies therein. The screening process comprises the steps of a sensitive tissue amyloid plaque immunoreactivity (TAPIR) assay on brain extracts for binding to ankG, and/or additional screening for binding on fragments, peptides or derivates of ankG and isolating the antibody for which binding is detected or the cell producing said antibody.

An immunoglobulin or its encoding cDNA may be further modified. Thus, in a further embodiment the method of the present invention comprises any one of the step(s) of producing a chimeric antibody, murinized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor (1988). When derivatives of said antibodies are obtained by the phage display technique, surface plasmon resonance as employed in the BIAcore system can be used to increase the efficiency of phage antibodies which bind to the same epitope as that of any one of the antibodies described herein (Schier, Human Antibodies Hybridomas 7 (1996), 97-105; Malmborg, J. Immunol. Methods 183 (1995), 7-13). The production of chimeric antibodies is described, for example, in international application WO89/09622. Methods for the production of humanized antibodies are described in, e.g., European application EP-A1 0 239 400 and international application WO90/07861. A further source of antibodies to be utilized in accordance with the present invention are so-called xenogeneic antibodies. The general principle for the production of xenogeneic antibodies such as human-like antibodies in mice is described in, e.g., international applications WO91/10741, WO94/02602, WO96/34096 and WO 96/33735. As discussed above, the antibody of the invention may exist in a variety of forms besides complete antibodies; including, for example, Fv, Fab and F(ab)₂, as well as in single chains; see e.g. international application WO88/09344.

As used herein, the term “sample” refers to any biological material obtained from a subject or patient. In one aspect, a sample can comprise blood, cerebrospinal fluid (“CSF”), or urine. In other aspects, a sample can comprise whole blood, plasma, B-cells enriched from blood samples, and cultured cells (e.g., B-cells from a subject). A sample can also include a biopsy or tissue sample including neural tissue. In still other aspects, a sample can comprise whole cells and/or a lysate of the cells. Blood samples can be collected by methods known in the art. In one aspect, the pellet can be resuspended by vortexing at 4° C. in 200 μl buffer (20 mM Tris, pH. 7.5, 0.5% Nonidet, 1 mM EDTA, 1 mM PMSF, 0.1M NaCl, IX Sigma Protease Inhibitor, and IX Sigma Phosphatase Inhibitors 1 and 2). The suspension can be kept on ice for 20 minutes with intermittent vortexing. After spinning at 15,000×g for 5 minutes at about 4° C., aliquots of supernatant can be stored at about −70° C.

As used herein, the terms “treat” or “treatment” refer to both therapeutic treatment and prophylactic or preventative measures, wherein the object is to prevent or slow down (lessen) an undesired physiological change or disorder, such as the development of Alzheimer's disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the manifestation of the condition or disorder is to be prevented.

If not stated otherwise the term “drug,” “medicine,” or “medicament” are used interchangeably herein and shall include but are not limited to all (A) articles, medicines and preparations for internal or external use, and any substance or mixture of substances intended to be used for diagnosis, cure, mitigation, treatment, or prevention of disease of either man or other animals; and (B) articles, medicines and preparations (other than food) intended to affect the structure or any function of the body of man or other animals; and (C) articles intended for use as a component of any article specified in clause (A) and (B). The term “drug,” “medicine,” or “medicament” shall include the complete formula of the preparation intended for use in either man or other animals containing one or more “agents,” “compounds”, “substances” or “(chemical) compositions” as and in some other context also other pharmaceutically inactive excipients as fillers, disintegrants, lubricants, glidants, binders or ensuring easy transport, disintegration, disaggregation, dissolution and biological availability of the “drug,” “medicine,” or “medicament” at an intended target location within the body of man or other animals, e.g., at the skin, in the stomach or the intestine. The terms “agent,” “compound” or “substance” are used interchangeably herein and shall include, in a more particular context, but are not limited to all pharmacologically active agents, i.e. agents that induce a desired biological or pharmacological effect or are investigated or tested for the capability of inducing such a possible pharmacological effect by the methods of the present invention.

By “subject” or “individual” or “animal” or “patient” or “mammal,” is meant any subject, particularly a mammalian subject, e.g., a human patient, for whom diagnosis, prognosis, prevention, or therapy is desired.

### DETAILED DESCRIPTION OF THE PRESENT INVENTION

Abnormal extracellular and intracellular deposits including the amyloid-β peptide (Aβ) and tau protein underlie the severe brain pathology in Alzheimer's disease (AD) and can induce an AD-related antigen immune response. The present invention is based on the observation of an abnormal extracellular presence of the neuronal cytoskeletal protein ankyrinG (ankG), present both in β-amyloid plaques and exosomal vesicles. Also an antibody response against ankG was observed which was higher in AD sera than in healthy control subjects (HCS) sera. Additionally, neuronal ankG expression was higher in AD patients than in age-matched healthy control subjects (HCS). Active immunization of APP-transgenic mice with ankG reduced brain β-amyloid pathology and antibodies against ankG reduced Aβ-induced loss of dendritic spines in ex vivo cultures. Furthermore, an interaction between ankG and APP was found. Moreover, after ankG silencing a decreased APP cell-surface trafficking and a lower Aβ production could be observed. The findings of the present invention, which are described in detail below and in the Examples, establish a surprising role for ankG in β-amyloid clearance and in APP metabolism. AnkG immunotherapy provides a novel avenue for Aβ-lowering therapy.

International application WO 2005/098433 describes experiments with human tissue sections from normal and Alzheimer disease patient donors as well as from the APP23 mouse model over-expressing human APP with the familial ‘Swedish’ double mutations suggesting that ankG is expressed in plaque-associated dystrophic neurites. However, though these observations led to the suggestion that during the pathogenesis of AD the neuronal distribution or subcellular localization of ankG is changed, further expanded studies were demanded in order to possibly establish whether the presence of ankG correlates with AD and disease progression. Accordingly, the role of ankG as a putative positive or negative marker of AD was still to be established. In this context, international application WO 2007/140973 describes that another member of the Ankyrin family, Ankyrin 2 was found at a decreased level in AD patients suggesting to reflect cytoskeletal impairment of neuronal cells in AD, which may result in a deficit in axonal transporting and neuritic dystrophy.

In contrast, experiments performed in accordance with the present invention revealed an abnormal extracellular presence of the neuronal cytoskeletal ankG in β-amyloid plaques and exosomal vesicles and a higher neuronal ankG expression in AD patients compared to age-matched healthy control subjects. Moreover, the experiments performed in accordance with the present invention revealed ankG as suitable target in the immunotherapy of amyloidosis and other neurodegenerative diseases which are due to abnormal aggregation of proteins.

Therefore, as a new means in the treatment of neurological disorders, the present invention relates to an agent capable of reducing the level of Ankyrin G (ankG), binding to ankG and/or interfering with the binding of ankG to a target protein in the brain for use in the treatment, amelioration or prevention of a neurological disorder, which agent will be referred to as “agent of the present invention” in further description. Put in other words, the present invention generally relates to ankG and derivatives thereof including any agent capable of interfering with the biological activity of ankG as a medicament useful in the treatment of neurological disorders. Regarding the mode of action, the present invention relates to any agent that could interfere with or prevent the (pathological) extracellular distribution and/or localisation of ankyrin G. In this context, the agent may interfere with ankG in the sense of reducing the level of expression of ankG, preventing ankG from binding to the target protein, for example by sequestering ankG and/or blocking its target protein binding domain, and the like. Likewise, ankG and an agent directly derived thereof such as peptides derived from ankG may compete with the binding of native ankG to its target protein. Thus, any agent which is capable of preventing the interaction of native ankG with its target protein in the brain is included within the definition of term “agent” according to the present invention. Furthermore, an “agent” of the present invention may also be referred to as “modulator”, i.e. a substance modulating the expression, activity or level of ankG. In accordance with the present invention modulation means a decrease (inhibition) in gene expression or amount, respective activity of ankG for which reason the agent may also be referred to as “antagonist” of ankG.

The term “ankG/ANKG” is also used to generally identify other conformers of ankG/ANKG, for example, oligomers or aggregates of ankG/ANKG. The term “ankG/ANKG” is also used to refer collectively to all types and forms of ankG/ANKG/Ank3/ANK3.

Two ankG/ANKG-isoforms are annotated in human. The amino acid sequence of ankG/ANKG isoform-1 of 4377aa can be retrieved from the literature and pertinent databases; see, e.g., Databank UniProtKB/Swiss-Prot: locus Q12955 (ANK3 HUMAN); Kordeli et al., J. Biol. Chem. 270 (1995), 2352-2359.

Said amino acid sequence of ANKG isoform-1 of 4377aa is:

(SEQ ID NO: 1)
MAHAASQLKKNRDLEINAEEEPEKKRKHRKRSRDRKKKSDANASYLRAARAGHLEKALDYIKNGVDIN
ICNQNGLNALHLASKEGHVEVVSELLQREANVDAATKKGNTALHIASLAGQAEVVKVLVTNGANVNAQ
SQNGFTPLYMAAQENHLEVVKFLLDNGASQSLATEDGFTPLAVALQQGHDQVVSLLLENDTKGKVRLP
ALHIAARKDDTKAAALLLQNDNNADVESKSGFTPLHIAAHYGNINVATLLLNRAAAVDFTARNDITPL
HVASKRGNANMVKLLLDRGAKIDAKTRDGLTPLHCGARSGHEQVVEMLLDRAAPILSKTKNGLSPLHM
ATQGDHLNCVQLLLQHNVPVDDVTNDYLTALHVAAHCGHYKVAKVLLDKKANPNAKALNGFTPLHIAC
KKNRIKVMELLLKHGASIQAVTESGLTPIHVAAFMGHVNIVSQLMHHGASPNTTNVRGETALHMAARS
GQAEVVRYLVQDGAQVEAKAKDDQTPLHISARLGKADIVQQLLQQGASPNAATTSGYTPLHLSAREGH
EDVAAFLLDHGASLSITTKKGFTPLHVAAKYGKLEVANLLLQKSASPDAAGKSGLTPLHVAAHYDNQK
VALLLLDQGASPHAAAKNGYTPLHIAAKKNQMDIATTLLEYGADANAVTRQGIASVHLAAQEGHVDMV
SLLLGRNANVNLSNKSGLTPLHLAAQEDRVNVAEVLVNQGAHVDAQTKMGYTPLHVGCHYGNIKIVNF
LLQHSAKVNAKTKNGYTPLHQAAQQGHTHIINVLLQNNASPNELTVNGNTALGIARRLGYISVVDTLK
IVTEETMTTTTVTEKHKMNVPETMNEVLDMSDDEVRKANAPEMLSDGEYISDVEEGEDAMTGDTDKYL
GPQDLKELGDDSLPAEGYMGFSLGARSASLRSFSSDRSYTLNRSSYARDSMMIEELLVPSKEQHLTFT
REFDSDSLRHYSWAADTLDNVNLVSSPIHSGFLVSFMVDARGGSMRGSRHHGMRIIIPPRKCTAPTRI
TCRLVKRHKLANPPPMVEGEGLASRLVEMGPAGAQFLGPVIVEIPHFGSMRGKERELIVLRSENGETW
KEHQFDSKNEDLTELLNGMDEELDSPEELGKKRICRIITKDFPQYFAVVSRIKQESNQIGPEGGILSS
TTVPLVQASFPEGALTKRIRVGLQAQPVPDEIVKKILGNKATFSPIVTVEPRRRKFHKPITMTIPVPP
PSGEGVSNGYKGDTTPNLRLLCSITGGTSPAQWEDITGTTPLTFIKDCVSFTTNVSARFWLADCHQVL
ETVGLATQLYRELICVPYMAKFVVFAKMNDPVESSLRCFCMTDDKVDKTLEQQENFEEVARSKDIEVL
EGKPIYVDCYGNLAPLTKGGQQLVFNFYSFKENRLPFSIKIRDTSQEPCGRLSFLKEPKTTKGLPQTA
VCNLNITLPAHKKETESDQDDEIEKTDRRQSFASLALRKRYSYLTEPGMIERSTGATRSLPTTYSYKP
FFSTRPYQSWTTAPITVPGPAKSGFTSLSSSSSNTPSASPLKSIWSVSTPSPIKSTLGASTTSSVKSI
SDVASPIRSFRTMSSPIKTVVSQSPYNIQVSSGTLARAPAVTEATPLKGLASNSTFSSRTSPVTTAGS
LLERSSITMTPPASPKSNINMYSSSLPFKSIITSAAPLISSPLKSVVSPVKSAVDVISSAKITMASSL
SSPVKQMPGHAEVALVNGSISPLKYPSSSTLINGCKATATLQEKISSATNSVSSVVSAATDTVEKVFS
TTTAMPFSPLRSYVSAAPSAFQSLRTPSASALYTSLGSSISATTSSVTSSIITVPVYSVVNVLPEPAL
KKLPDSNSFTKSAAALLSPIKTLTTETHPQPHFSRTSSPVKSSLFLAPSALKLSTPSSLSSSQEILKD
VAEMKEDLMRMTAILQTDVPEEKPFQPELPKEGRIDDEEPFKIVEKVKEDLVKVSEILKKDVCVDNKG
SPKSPKSDKGHSPEDDWIEFSSEEIREARQQAAASQSPSLPERVQVKAKAASEKDYNLTKVIDYLTND
IGSSSLTNLKYKFEDAKKDGEERQKRVLKPAIALQEHKLKMPPASMRTSTSEKELCKMADSFFGTDTI
LESPDDFSQHDQDKSPLSDSGFETRSEKTPSAPQSAESTGPKPLFHEVPIPPVITETRTEVVHVIRSY
DPSAGDVPQTQPEEPVSPKPSPTFMELEPKPTTSSIKEKVKAFQMKASSEEDDHNRVLSKGMRVKEET
HITITTRMVYHSPPGGEGASERIEETMSVHDIMKAFQSGRDPSKELAGLFEHKSAVSPDVHKSAAETS
AQHAEKDNQMKPKLERIIEVHIEKGNQAEPTEVIIRETKKHPEKEMYVYQKDLSRGDINLKDFLPEKH
DAFPCSEEQGQQEEEELTAEESLPSYLESSRVNTPVSQEEDSRPSSAQLISDDSYKTLKLLSQHSIEY
HDDELSELRGESYRFAEKMLLSEKLDVSHSDTEESVTDHAGPPSSELQGSDKRSREKIATAPKKEILS
KIYKDVSENGVGKVSKDEHFDKVTVLHYSGNVSSPKHAMWMRFTEDRLDRGREKLIYEDRVDRTVKEA
EEKLTEVSQFFRDKTEKLNDELQSPEKKARPKNGKEYSSQSPTSSSPEKVLLTELLASNDEWVKARQH
GPDGQGFPKAEEKAPSLPSSPEKMVLSQQTEDSKSTVEAKGSISQSKAPDGPQSGFQLKQSKLSSIRL
KFEQGTHAKSKDMSQEDRKSDGQSRIPVKKIQESKLPVYQVFAREKQQKAIDLPDESVSVQKDFMVLK
TKDEHAQSNEIVVNDSGSDNVKKQRTEMSSKAMPDSFSEQQAKDLACHITSDLATRGPWDKKVFRTWE
SSGATNNKSQKEKLSHVLVHDVRENHIGHPESKSVDQKNEFMSVTERERKLLTNGSLSEIKEMTVKSP
SKKVLYREYVVKEGDHPGGLLDQPSRRSESSAVSHIPVRVADERRMLSSNIPDGFCEQSAFPKHELSQ
KLSQSSMSKETVETQHFNSIEDEKVTYSEISKVSKHQSYVGLCPPLEETETSPTKSPDSLEFSPGKES
PSSDVFDHSPIDGLEKLAPLAQTEGGKEIKTLPVYVSFVQVGKQYEKEIQQGGVKKIISQECKTVQET
RGTFYTTRQQKQPPSPQGSPEDDTLEQVSFLDSSGKSPLTPETPSSEEVSYEFTSKTPDSLIAYIPGK
PSPIPEVSEESEEEEQAKSTSLKQTTVEETAVEREMPNDVSKDSNQRPKNNRVAYIEFPPPPPLDADQ
IESDKKHHYLPEKEVDMIEVNLQDEHDKYQLAEPVIRVQPPSPVPPGADVSDSSDDESIYQPVPVKKY
TFKLKEVDDEQKEKPKASAEKASNQKELESNGSGKDNEFGLGLDSPQNEIAQNGNNDQSITECSIATT
AEFSHDTDATEIDSLDGYDLQDEDDGLTESDSKLPIQAMEIKKDIWNTEGILKPADRSFSQSKLEVIE
EEGKVGPDEDKPPSKSSSSEKTPDKTDQKSGAQFFTLEGRHPDRSVFPDTYFSYKVDEEFATPFKTVA
TKGLDFDPWSNNRGDDEVFDSKSREDETKPFGLAVEDRSPATTPDTTPARTPTDESTPTSEPNPFPFH
EGKMFEMTRSGAIDMSKRDFVEERLQFFQIGEHTSEGKSGDQGEGDKSMVTATPQPQSGDTTVETNLE
RNVETPTVEPNPSIPTSGECQEGTSSSGSLEKSAAATNTSKVDPKLRTPIKMGISASTMTMKKEGPGE
ITDKIEAVMTSCQGLENETITMISNTANSQMGVRPHEKHDFQKDNFNNNNNLDSSTIQTDNIMSNIVL
TEHSAPTCTTEKDNPVKVSSGKKTGVLQGHCVRDKQKVLGEQQKTKELIGIRQKSKLPIKATSPKDTF
PPNHMSNTKASKMKQVSQSEKTKALTTSSCVDVKSRIPVKNTHRDNIIAVRKACATQKQGQPEKGKAK
QLPSKLPVKVRSTCVTTTTTTATTTTTTTTTTTTSCTVKVRKSQLKEVCKHSIEYFKGISGETLKLVD
RLSEEEKKMQSELSDEEESTSRNTSLSETSRGGQPSVTTKSARDKKTEAAPLKSKSEKAGSEKRSSRR
TGPQSPCERTDIRMAIVADHLGLSWTELARELNFSVDEINQIRVENPNSLISQSFMLLKKWVTRDGKN
ATTDALTSVLTKINRIDIVTLLEGPIFDYGNISGTRSFADENNVFHDPVDGWQNETSSGNLESCAQAR
RVTGGLLDRLDDSPDQCRDSITSYLKGEAGKFEANGSHTEITPEAKTKSYFPESQNDVGKQSTKETLK
PKIHGSGHVEEPASPLAAYQKSLEETSKLIIEETKPCVPVSMKKMSRTSPADGKPRLSLHEEEGSSGS
EQKQGEGFKVKTKKEIRHVEKKSHS

A further isoform, ankG/ANKG isoform-2 of 1001aa, is annotated in Human with the Databank accession number: B1AQT2 (B1AQT2_HUMAN); Kapfhamer et al., Genomics 27 (1), 189-191 (1995); Lee et al., Mol. Psychiatry. 2010 Apr. 13. [Epub ahead of print]; Williams et al., Hum. Mol. Genet. 20 (2), 387-391 (2011).

Said amino acid sequence of ANKG isoform-2 of 1001aa is:

(SEQ ID NO: 2)
MALPQSEDAMTGDTDKYLGPQDLKELGDDSLPAEGYMGFSLGARSASLRSFSSDRSYTLNRS
SYARDSMMIEELLVPSKEQHLTFTREFDSDSLRHYSWAADTLDNVNLVSSPIHSGFLVSFMV
DARGGSMRGSRHHGMRIIIPPRKCTAPTRITCRLVKRHKLANPPPMVEGEGLASRLVEMGPA
GAQFLGPVIVEIPHFGSMRGKERELIVLRSENGETWKEHQFDSKNEDLTELLNGMDEELDSP
EELGKKRICRIITKDFPQYFAVVSRIKQESNQIGPEGGILSSTTVPLVQASFPEGALTKRIR
VGLQAQPVPDEIVKKILGNKATFSPIVTVEPRRRKFHKPITMTIPVPPPSGEGVSNGYKGDT
TPNLRLLCSITGGTSPAQWEDITGTTPLTFIKDCVSFTTNVSARFWLADCHQVLETVGLATQ
LYRELICVPYMAKFVVFAKMNDPVESSLRCFCMTDDKVDKTLEQQENFEEVARSKDIEVLEG
KPIYVDCYGNLAPLTKGGQQLVFNFYSFKENRLPFSIKIRDTSQEPCGRLSFLKEPKTTKGL
PQTAVCNLNITLPAHKKIEKTDRRQSFASLALRKRYSYLTEPGMSPQSPCERTDIRMAIVAD
HLGLSWTELARELNFSVDEINQIRVENPNSLISQSFMLLKKWVTRDGKNATTDALTSVLTKI
NRIDIVTLLEGPIFDYGNISGTRSFADENNVFHDPVDGYPSLQVELETPTGLHYTPPTPFQQ
DDYFSDISSIESPLRTPSRLSDGLVPSQGNIEHSADGPPVVTAEDASLEDSKLEDSVPLTEM
PEAVDVDESQLENVCLSWQNETSSGNLESCAQARRVTGGLLDRLDDSPDQCRDSITSYLKGE
AGKFEANGSHTEITPEAKTKSYFPESQNDVGKQSTKETLKPKIHGSGHVEEPASPLAAYQKS
LEETSKLIIEETKPCVPVSMKKMSRTSPADGKPRLSLHEEEGSSGSEQKQGEGFKVKTKKEI
RHVEKKSHS

Further Ankyrin G variants and splicing isoforms in human and other organisms such as mouse are known to the person skilled in the art. Their nucleotide and amino acid sequences can be retrived from known databases such as UniProtKB/SwissProt/TrEMBL, GenBank, RefSeq, TPA, PIR, PRF and PDB in which context also the above mentioned synonymes for AnkyrinG may be used. Examples for such sequences are annotated in the mouse with the Databank accession numbers UniProtKB/TrEMBL: Q4U260_MOUSE, Q4U205_MOUSE, Q8VC68_MOUSE, Q8CBN3_MOUSE, Q3TSJ8_MOUSE; GenBank: NP 666117.2, NP_{—}733791.2, NP_{—}733925.2 and further published in Okazaki et al., Nature 420 (2002), 563-573, Carninci and Hayashizaki, Methods Enzymol. 303 (1999), 19-44, Hoock et al., J. Cell Biol. 136 (1997), 1059-1070. Further examples of human Ankyrin G variants are Q5JSX5_HUMAN, Q13484_HUMAN, Q59G01_HUMAN and A8KA62_HUMAN; see also Devarajan et al., J. Cell Biol. 133 (1996), 819-830.

In view of the data presented in the examples of the present invention, the agent of the present invention is preferably used in the treatment, amelioration or prevention of a neurological disorder, wherein the disorder is associated with Alzheimer's disease. Furthermore, the disorder is preferably associated with amyloid β (Aβ) pathology/amyloidosis.

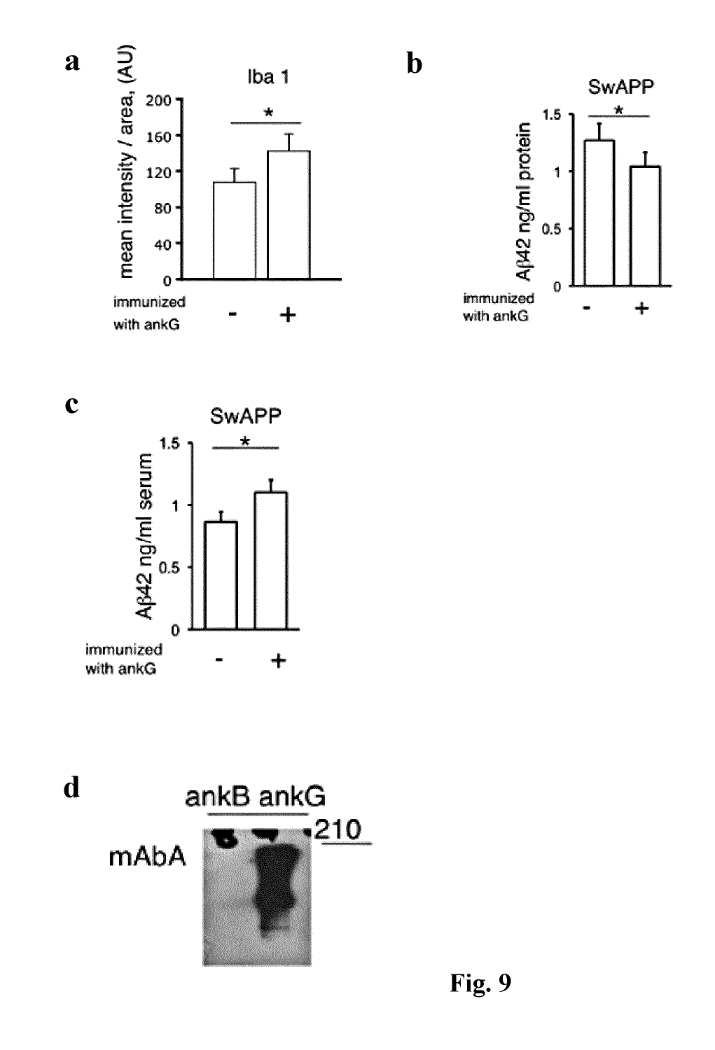

As described in the Examples, active immunization with ankG reduces β-amyloid pathology, i.e. the number and size of β-amyloid plaques and Aβ42 brain levels in Alzheimer's disease model mice; see Example 3 and FIGS. 4a, 4b and 9b. Therefore, in one preferred embodiment of the present invention said agent is recombinant ankG protein or a fragment, derivative or analog thereof.

Aβ is a hydrophobic peptide and thus the presence of ankG in β-amyloid plaques could be due to a non-specific interaction with hydrophobic ankG domains. Quite the contrary, as shown in Example 5, no interaction could be found between ankG and Aβ. However, APP but not Aβ could be shown to interact with ankG in co-immunoprecipitation experiments; see Example 5 and FIG. 6a. Thus, according to one embodiment of the present invention the agent of the present invention is capable of interfering with the interaction of ankG and amyloid precursor protein (APP). In a further embodiment of the present invention, the agent of the present invention is capable of detecting and/or binding APP/ankG complexes.

The agents which are used according to the present invention, may be of different kind. In one embodiment the agent of the present invention is an ankG-binding molecule. As shown in the examples, antibodies against ankG lower Aβ levels and Aβ-induced spine loss in hippocampal cultures from said model animals, i.e. from arcAbeta mice; see Example 4 and FIGS. 5 a-c. Thus, in one preferred embodiment of the present invention said ankG-binding molecule is an anti-ankG antibody.

As already mentioned supra, APP interacts with ankG in immunoprecipitation experiments; see also Example 5. Therefore, in another embodiment of the present invention said ankG-binding molecule is a fragment, derivative or analog of APP, preferably wherein said fragment, derivative or analog is derived from the intracytoplasmatic domain of APP.

As shown in Example 3 of the present invention, active immunization by introduction of peptides or peptide epitopes, i.e. vaccination, into a subject can elicit similar effects as passive immunization, i.e. provision of antibodies specifically binding said peptides or peptide epitopes; see Example 4. In this context, the person skilled in the art is well aware of the fact that in the manufacture of vaccines, in particular against endogenous peptides or peptide epitopes the purity of the pathological structure used as the antigen is of particular relevance, since any impurity may result in undesired immune reactions, which can manifested themselves in the form of auto-immune-diseases up to septic shock with even lethal consequences. In particular, for preventive medical treatment but also for therapy the safety of a drug is a key criterion, for their development.

Therefore, in one embodiment of the present invention said agent is formulated as a vaccine. Preferably, said vaccine is capable of inducing autoantibodies against ankG.

For use in vaccination said agent has to be formulated, i.e. compositions of the active agent, e.g. a recombinant peptide or fragment of ankG, with adjuvants has to be produced in such a way as to enhance the bioavailability of the vaccine and the immunological response against it. Almost all adjuvants used today for enhancement of the immune response against antigens are particles or are forming particles together with the antigen, see also “Vaccine Design—the subunit and adjuvant approach” (Ed: Powell & Newman, Plenum Press, 1995) for details. The present invention also relates to a composition for treating a disease, in particular neurodegenerative, neurological or neuropsychiatric disorder comprising the recombinant peptide or fusion protein, as an active agent as described above, and optionally a pharmaceutically acceptable carrier. Similarly, compositions containing peptides, polypeptides or fusion proteins binding, e.g., ankG and pharmaceutically acceptable carriers may be used for diagnosing of said neurodegenerative, neurological or neuropsychiatric disorders.

Pharmaceutically acceptable carriers and administration routes can be taken from corresponding literature known to the person skilled in the art. The pharmaceutical compositions of the present invention can be formulated according to methods well known in the art; see for example Remington: The Science and Practice of Pharmacy (2000) by the University of Sciences in Philadelphia, ISBN 0-683-306472, Vaccine Protocols. 2nd Edition by Robinson et al., Humana Press, Totowa, N.J., USA, 2003; Banga, Therapeutic Peptides and Proteins: Formulation, Processing, and Delivery Systems. 2nd Edition by Taylor and Francis. (2006), ISBN: 0-8493-1630-8. Examples of suitable pharmaceutical carriers are well known in the art and include phosphate buffered saline solutions, water, emulsions, such as oil/water emulsions, various types of wetting agents, sterile solutions etc. Preferably, the pharmaceutically acceptable carrier is KLH, tetanus toxoid, albumin binding protein, bovine serum albumin, or an adjuvant substance described in Singh et al., Nat. Biotech. 17 (1999), 1075-1081 and O'Hagan et al., Nature Reviews, Drug Discovery 2 (9) (2003), 727-735, or mixtures thereof. In addition, the vaccine composition may contain aluminium hydroxyde. Compositions comprising such carriers can be formulated by well known conventional methods. These pharmaceutical compositions can be administered to the subject at a suitable dose. Administration of the suitable compositions may be effected by different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. Aerosol formulations such as nasal spray formulations include purified aqueous or other solutions of the active agent with preservative agents and isotonic agents. Such formulations are preferably adjusted to a pH and isotonic state compatible with the nasal mucous membranes.

Formulations for rectal or vaginal administration may be presented as a suppository with a suitable carrier; see also O'Hagan et al., Nature Reviews, Drug Discovery 2(9) (2003), 727-735. Further guidance regarding formulations that are suitable for various types of administration can be found in Remington's Pharmaceutical Sciences, Mace Publishing Company, Philadelphia, Pa., 17th ed. (1985) and corresponding updates. For a brief review of methods for drug delivery see Langer, Science 249 (1990), 1527-1533.

The peptides, polypeptides or fusion proteins may be provided by expression in a host cell, for example. To express the peptide, polypeptide or fusion protein in a host cell, the nucleic acid molecule encoding said peptide, polypeptide or fusion protein may be inserted into appropriate expression vector, i.e. a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989); see also the literature cited in the Examples section.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

To express the peptide, polypeptide or fusion protein (hereinafter referred to as “product”) in a host cell, a procedure such as the following can be used. A restriction fragment containing a DNA sequence that encodes said product may be cloned into an appropriate recombinant plasmid containing an origin of replication that functions in the host cell and an appropriate selectable marker. The plasmid may include a promoter for inducible expression of the product (e.g., pTrc (Amann et al, Gene 69 (1988), 301 315) and pET1 Id (Studier et al., Gene Expression Technology Methods in Enzymology 185, Academic Press, San Diego, Calif. (1990), 60 89). The recombinant plasmid may be introduced into the host cell by, for example, electroporation and cells containing the recombinant plasmid may be identified by selection for the marker on the plasmid. Expression of the product may be induced and detected in the host cell using an assay specific for the product.

A suitable host cell for expression of the product may be any prokaryotic or eukaryotic cell; e.g., bacterial cells such as E. coli or B. subtilis, insect cells (baculovirus), yeast, or mammalian cells such as Chinese hamster ovary cell (CHO). In some embodiments, the DNA that encodes the product/peptide may be optimized for expression in the host cell. For example, the DNA may include codons for one or more amino acids that are predominant in the host cell relative to other codons for the same amino acid.

Alternatively, the expression of the product may be performed by in vitro synthesis of the protein in cell-free extracts which are also particularly suited for the incorporation of modified or unnatural amino acids for functional studies; see also infra. The use of in vitro translation systems can have advantages over in vivo gene expression when the over-expressed product is toxic to the host cell, when the product is insoluble or forms inclusion bodies, or when the protein undergoes rapid proteolytic degradation by intracellular proteases. The most frequently used cell-free translation systems consist of extracts from rabbit reticulocytes, wheat germ and Escherichia coli. All are prepared as crude extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract must be supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg²⁺, K⁺, etc.). Appropriate transcription/translation systems are commercially available, for example from Promega Corporation, Roche Diagnostics, and Ambion, i.e. Applied Biosystems.

Since the agent of the present invention is preferably used in the treatment, amelioration or prevention of a neurological disorder as described above, in a further preferred embodiment of the present invention an agent is provided, wherein said agent is capable of reducing the expression or the cortical level of ankG protein in the brain.

Expression of genes or levels of specific proteins in cells or organs can be reduced by techniques using antisense molecules, for example. “Antisense molecules” or “antisense reagents” can, in the present context, be any molecule that hybridizes by a sequence specific base pairing to a complementary DNA and/or RNA sequence. In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid nonspecific binding of the antisense compound to non-target sequences under conditions in which specific binding is desired, i.e., under physiological conditions in the case of in vivo assays, and in the case of in vitro assays, under conditions in which the assays are performed. Typical “antisense molecules” or “antisense reagents” are any oligonucleotide, such as DNA, RNA, any peptide nucleic acid, any other nucleic acid derivative, or mimic and/or derivative thereof. The target sequence is not restricted to the “sense” or “coding” strand of mRNA, although this is often the target. According to the present invention “antisense molecules,” or “antisense constructs” can be employed which are used interchangeably in the present text. In one embodiment of the present invention the use of oligonucleotides, for use in modulating the function of nucleic acid molecules encoding genes, in particular of the ankG gene is addressed. This is accomplished by providing antisense compounds which specifically hybridize with one or more nucleic acids encoding a target gene, such as the ankG gene.

As used herein, the term “target nucleic acid” encompasses a DNA encoding said gene, and/or an RNA (including pre-mRNA and mRNA) transcribed from such DNA. The specific hybridization of an oligomeric compound with its target nucleic acid interferes with the normal function of the nucleic acid. This modulation of function of a target nucleic acid by compounds which specifically hybridize to it is generally referred to as “antisense” (when the target is RNA) or “antigene” (when the target is DNA). The functions of DNA to be interfered with include replication and transcription. This effect is referred to as “antigene”. Such interactions may occure by binding of the “antigene” molecule to the DNA double-helix as a third strand in its major groove forming a structure also known as “triplex DNA” or “triple helix DNA” (Frank-Kamenetskii, Annu. Rev. of Biochem. 64 (1995), 65-95; Rusling et al., Nucleic Acids Res. 33 (2005), 3025-3032). The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA and is referred to as “antisense”. However, the distinction between “antisense” and “antigene” is not absolute.

The overall effect of such interferences with target nucleic acid function is a specific modulation of the expression of said essential gene. In the context of the present invention, “modulation” means either an increase (stimulation) or a decrease (inhibition) in the expression of a gene. In the context of the present invention, inhibition is the preferred form of modulation of gene expression.

In the present invention, antisense molecules can be selected from the group consisting of oligonucleotides, oligonucleotide analogues, oligonucleotide mimics, such as for example PNA, locked nucleic acids (LNA), phosphorothioate, 2′-methoxy-, 2′-methoxyethoxy-, morpholino, phosphoramidate oligonucleotides or the like. In the present invention, antigene molecules can furthermore be selected from the group consisting of triplex forming or strand invading oligonucleotides, oligonucleotide analogues, oligonucleotide mimics, such as for example PNA, locked nucleic acids (LNA), phosphorothioate, 2′-methoxy-, 2′-methoxyethoxy-, morpholino, phosphoramidate oligonucleotides or DNA minor groove binding polyamides (oligo pyrroles/imidazoles etc.) as described (Gottesfeld et al., Gene Expr. 9 (2000), 77-91; Dervan and Bürli, Curr. Opin. Chem. Biol. 3 (1999), 688-693) or the like.

Peptide nucleic acid (PNA) is an antisense compound that has shown good results for specific gene targeting in a number of different organisms (Good and Nielsen, Antisense Nucleic Acid Drug Dev. 7 (1997), 431-437; Larsen et al., Biochim. Biophys. Acta. 1489 (1999), 159-166; Nielsen et al., Science 254 (1991), 1497-1500, WO 93/12129, U.S. Pat. No. 5,773,571). In PNA compounds, the sugar backbone of an oligonucleotide is replaced with an amide containing backbone, in particular with an aminoethylglycine backbone. The nucleobases are retained and are bound directly or indirectly to aza nitrogen atoms of the amide portion of the backbone.

Representative US patents that teach the preparation of PNA compounds include U.S. Pat. No. 5,539,082; U.S. Pat. No. 5,714,331; and U.S. Pat. No. 5,719,262, each of which is herein incorporated by reference. Further teaching of PNA compounds can be found in Nielsen et al., Science. 254 (1991), 1497-1500. In addition, the use of PNA oligomers as anti-sense oligomers for the treatment of diseases is taught by WO 93/12129 and U.S. Pat. No. 5,773,571; both are incorporated herein by reference

Locked Nucleic Acid (LNA) are a bi-cyclic DNA analogue which may also be used as oligonucleotide mimetics (see International Patent Application WO 99/14226; Nielsen et al., J. Chem. Soc., Perkin Trans., 1 (1997), 3423-3433; Nielsen et al., Chem. Commun., 9 (1997), 825-826; Christensen et al., J. Am. Chem. Soc., 120 (1998), 5458-5463; Koshkin et al., J. Org. Chem., 63 (1998), 2778-2781; Koshkin et al., J. Am. Chem. Soc. 120 (1998), 13252-13253; Kumar et al., Bioorg. Med. Chem. Lett., 8 (1998), 2219-2222; and Obika et al., Bioorg. Med. Chem. Lett., 9 (1999), 515-518).

The term “oligonucleotide(s)” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally occurring nucleobases, sugars and covalent internucleoside (backbone) linkages which function similarly or combinations thereof. Such modified or substituted oligonucleotides are often preferred over native forms because of desirable properties such as, for example, enhanced cellular uptake, enhanced affinity for nucleic acid target and increased stability in the presence of nucleases and other enzymes, and are in the present context described by the terms “oligonucleotide analogues” or “oligonucleotide mimics”.

The antisense compounds in accordance with this invention preferably comprise from 7 to 80 nucleobase units, preferably not more than 30 nucleobase units to avoid an interferon response (Manche et al., Mol. Cell. Biol. 12 (1992), 5238-5248). The term “nucleobase units” is used in the present text to describe both the number of nucleotides in an oligonucleotide and the number of nucleobase-carrying monomers of an oligonucleotide mimetic. Particularly preferred antisense compounds are antisense oligonucleotides, even more preferably those comprising from 14 to 29 nucleobases. Most preferred are short RNA based antisense oligonucleotides comprising around 20 nucleobases, i.e. from 18 to 26 nucleobases, of two particular molecular classes, either single stranded (miRNA) or double stranded (siRNA).

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′,3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

A further class of agents which may be used according to the present invention to lower the expression or protein levels of ankG are ribozymes. The term “Ribozyme” refers to a nucleic acid molecule which is capable of cleaving a specific nucleic acid sequence. Ribozymes may be composed of RNA, DNA, nucleic acid analogues (e.g., phosphorothioates), or any combination of these (e.g., DNA/RNA chimerics). Within particularly preferred embodiments, a ribozyme should be understood to refer to RNA molecules that contain anti-sense sequences for specific recognition, and an RNA-cleaving enzymatic activity. “Ribozyme gene” refers to a nucleic acid molecule (e.g., DNA) consisting of the ribozyme sequence which, when transcribed into RNA, will yield the ribozyme.

Several different types of ribozymes may be constructed for use within the present invention, including for example, hammerhead ribozymes (Rossi et al., Pharmac. Ther. 50 (1991), 245-254; Forster and Symons, Cell 49 (1987), 211-220; Uhlenbeck, Nature 328 (1988), 596-600; Walbot and Bruening, Nature 334 (1988), 196-197; Haseloff and Gerlach, Nature 334 (1988), 585-591; U.S. Pat. No. 5,254,678), hairpin ribozymes (Hampel et al., Nucl. Acids Res. 18 (1990), 299-304, and U.S. Pat. No. 5,254,678), hepatitis delta virus ribozymes (Perrotta and Been, Biochem. 31 (1992), 16-21,), Group I intron ribozymes (U.S. Pat. No. 4,987,071) and RNase P ribozymes (Guerrier-Takada et al., Cell 35 (1983), 849-857; WO 95/31551)

U.S. Pat. No. 4,987,071 has disclosed the preparation and use of ribozymes which are based on the properties of the Tetrahymena ribosomal RNA self-splicing reaction. These ribozymes require an eight base pair target site and free guanosine (or guanosine derivatives). A temperature optimum of 50° C. is reported for the endoribonuclease activity. The fragments that arise from cleavage contain 5′-phosphate and 3′-hydroxyl groups and a free guanosine nucleotide added to the 5′-end of the cleaved RNA.

In contrast to the ribozymes as described in U.S. Pat. No. 4,987,071, particularly preferred ribozymes of the present invention hybridize efficiently to target sequences at physiological temperatures, making them suitable for use in vivo, and not merely as research tools (see column 15, lines 18 to 42, of U.S. Pat. No. 4,987,071). Thus, particularly preferred ribozymes for use within the present invention include hairpin ribozymes (for example, as described in EP 0 360 257) and hammerhead ribozymes.

Unmodified, naked antisense molecules were reported to be internalized poorly by cells, whether or not they are negatively charged (Grey et al., Biochem. Pharmacol. 53 (1997). 1465-1476, Stein et al., Biochemistry 32 (1993), 4855-4861. Bennet et al., Mol. Pharmacol. 41 (1992), 1023-1033). Therefore, the oligonucleotides may be modified or used in compositions with other agents such as lipid carriers (Fattal et al., Adv. Drug Deliv. Rev. 56 (2004), 931-946), microparticles (Khan et al., J. Drug Target 12 (2004), 393-404) or by covalent conjugation to cell-penetrating peptides (CPP) allowing translocation of the antisense molecules through the cell membrane; see Lysik and Wu-Pong, J. Pharm. Sci. 92 (2003), 1559-1573 for an review

Modifications of the oligonucleotides, by chemically linking to the oligonucleotide one or more moieties or conjugates may enhance the activity, cellular distribution or cellular uptake of the oligonucleotide or may be used to label the oligonucleotides for in vitro or in vivo imaging. Such moieties include, but are not limited to, lipid moieties such as a cholesterol moiety (Letsinger et al., Proc. Natl. Acad. Sci. USA, 86 (1989), 6553-6556), cholic acid (Manoharan et al., Bioorg. Med. Chem. Let., 4 (1994), 1053-1060), a thioether, e.g., hexyl-S-tritylthiol (Manoharan et al., Ann. N.Y. Acad. Sci., 660 (1992), 306-309; Manoharan et al., Bioorg. Med. Chem. Let., 3 (1993), 2765-2770), a thiocholesterol (Oberhauser et al., Nucl. Acids Res., 20 (1992), 533-538), an aliphatic chain, e.g., dodecandiol or undecyl residues (Saison-Behmoaras et al., EMBO J. 10 (1991), 1111-1118; Kabanov et al., FEBS Lett. 259 (1990), 327-330; Svinarchuk et al., Biochimie 75 (1993), 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 36 (1995), 3651-3654; Shea et al., Nucl. Acids Res. 18 (1990), 3777-3783), a polyamine or a polyethylene glycol chain (Manoharan et al., Nucleosides & Nucleotides 14 (1995), 969-973), or adamantane acetic acid (Manoharan et al., Tetrahedron Lett., 36 (1995), 3651-3654), a palmityl moiety (Mishra et al., Biochim. Biophys. Acta, 1264 (1995), 229-237), or an octadecylamine or hexylamino-carbonyl-oxycholesterol moiety (Crooke et al., J. Pharmacol. Exp. Ther., 277 (1996), 923-937). For labeling of oligonucleotides radioactive, nonadioactive, fluorescent and enzymatic moieties are used such as, e.g., fluorescein, acridine, Digoxigenin (DIG), biotin (Schmitz et al., Anal. Biochem. 192 (1991), 222-231; Nelson et al., Nucl. Acids Res. 20 (1992), 6253-6259; van Gijlswijk et al., Expert Rev Mol. Diagn. 1 (2001), 81-91), 1,4,7,10-tetraazacyclododecane-1,4,7,10-tetraacetic acid (DOTA) (Jaakkola et al., Curr. Protoc. Nucleic Acid. Chem. 2007 June; Chapter 14:Unit 4.31) or radionuclides for imaging of gene expression with PET (Lendvei et al., Curr Med. Chem. 16 (2009), 4445-4461)

Representative US patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

Summarizing, the agent of the present invention may be also selected from one of the above described molecule classes reducing expression or protein levels of ankG, preferably wherein said agent is selected from the group consisting of triple helix DNA, antisense nucleic acid, microRNAs, double stranded RNA molecules and ribozymes, most preferably wherein an agent is selected which is a small interfering RNA (siRNA).

In another aspect, the present invention relates to a method for isolating an agent useful in the treatment of a neurological disorder, comprising the steps of (a) subjecting a test compound to ankG protein or a fragment thereof; and (b) determining whether the compound is capable of binding ankG protein or interfering with binding of ankG with APP or an ankG-binding fragment thereof, which is indicative for its usefulness as a drug. By said method isolated agent is useful in the treatment, amelioration or prevention of neurological disorder, preferably wherein the disorder is associated with Alzheimer's disease. Alternatively or in addition the agent isolated by said method is useful in treatment of a neurological disorder, wherein the disorder is associated with amyloid β (Aβ) pathology/amyloidosis.

Concerning the screening applications of the present invention relating to the testing of pharmaceutical compounds in drug research, it is generally referred to the standard textbook “In vitro Methods in Pharmaceutical Research”, Academic Press, 1997. In general, according to the present invention, the binding of ankG protein or interfering with binding of ankG with APP or an ankG-binding fragment thereof is indicative for a putative drug.

Several strategies have been described in the prior art to detect and monitor, respectively, binding between molecules, and as a consequence detecting inhibition or modulation of said binding, respectively, which may be used in accordance with the present invention. Those strategies comprise for example tagging at least one partner with molecules the properties of which change upon binding such as illuminating molecules, wherein the detected signal might be light emittance such as fluorescence increase or decrease, or gaining additional or loosing former properties upon binding. Those strategies may of course also be used in accordance with the present invention, i.e. to detect and control, respectively, binding or non-binding of the ankG to its interacting molecule or interfering with binding of ankG with APP or a fragment thereof.

For example, according to said method for isolating an agent useful in the treatment of a neurological disorder a compound can be tested, wherein said compound is capable of competing with the binding of APP to ankG. Furthermore, as described supra, one of the factors tested during said screening method is whether the compound is capable of binding ankG protein. For this purpose, binding of ankG or a fragment thereof is determined by an anti-ankG antibody in one embodiment of the present invention. In a further embodiment of the present invention, this antibody is detectably labeled or otherwise modified and/or to be detected by a secondary antibody.

The present invention also contemplates screens for small molecules, analogs thereof, as well as screens for natural ankG interacting molecules such as those that bind to and/or inhibit aggregation of ankG or interfere with binding of ankG with APP or an ankG-binding fragment thereof in vitro and/or in vivo. Suitable test agents for the screening methods may include antibodies, chelating agents, tridentate iron chelators, diketones, 2-pyridoxal isonicontinyl hydrazone analogues, tachypyridine, clioquinol, ribonucleotide reductase inhibitor chelators, 2,3-dihydroxybenzoic acid, Picolinaldehyde, Nicotinaldehyde, 2-Aminopyridine, 3-Aminopyridine, topical 2-furildioxime, n-Butyric acid, Phenylbutyrate, Tributyrin, suberoylanilide hydroxamic acid, 6-cyclohexyl-1-hydroxy-4-methyl-2(1H)-pyridinone, rilopirox, piroctone, benzoic acid-related chelators, salicylic acid, nicotinamide, Clioquniol, heparin sulfate, trimethylamine N-oxide, polyethylene glycol (PEG), copper cations, dimethylsulfoxide, Dexrazoxane, dopamine, tannic acid, triazine, levodopa, pergolide, bromocriptine, selegiline, glucosamine or analogs thereof, tetrapyrroles, nordihydroguaiaretic acid, polyphenols, tetracycline, polyvinylsulfonic acid, 1,3,-propanedisulfonic acid, β-sheet breaker peptide, e.g., 5-amino acid β-sheet breaker peptide (iAβ5P), nicotine, or salts or derivatives thereof to name a few.

In one embodiment of the present invention the contemplated screening method for isolating an agent useful in the treatment of a neurological disorder, compounds are tested wherein the compound is a peptide. In a further embodiment of the present invention, this peptide or ankG-binding fragment is derived from the intracytoplasmatic domain of APP, preferably wherein the peptide consists of about 10 to 75 amino acids.

In some embodiments concerning the method for isolation an agent useful in the treatment of a neurological disorder, both kinds of substances which have to be tested, i.e. (i) APP or an ankG-binding fragment thereof; or (ii) the compound to be screened is arranged on a solid support. This can be achieved by methods known in the art, such as methods comprising exposing a peptide to a solid support for a sufficient amount of time to permit immobilization of the probe to the solid support. The methods may further comprise removing unbound peptide, cross-linking the peptide to the solid support (e.g., chemically and/or by exposure to UV-irradiation), and drying the solid support and peptide. In an alternative embodiment, a peptide-based array may be used, which is for example loaded with hydrophobic peptides of the present invention in order to detect autoantibodies which may be present in patients suffering from a disease such as a neurological disorder, in particular Alzheimer's disease. For example, antigen microarray profiling of autoantibodies in rheumatoid arthritis has been reported by Hueber et al., Arthritis Rheum. 52 (2005), 2645-2655. Design of microarray immunoassays is summarized in Kusnezow et al., Mol. Cell. Proteomics 5 (2006), 1681-1696.

As shown in the examples, the recombinant ankG protein and fragments thereof of the present invention hold great promise in the generation of vaccines against AD and other Aβ associated diseases as well as in the use as a means for the detection of the risk of disease, diagnosis of disease, and disease progression and etiology. The following is a non-limiting list of Aβ associated diseases: Alzheimer's disease, Down's syndrome, Lewy body dementia, head trauma, dementia pugilistica, amyloid deposition associated with aging, mild cognitive impairment, vascular dementia, mixed dementia, hereditary cerebral hemorrhage with amyloidosis Dutch type and Icelandic type, glaucoma, Parkinson's disease, frontotemporal dementia, corticobasal degeneration, inclusion body myositis and cerebral amyloid angiopathy.

Concerning the detection or diagnosis of a neurological diseases, the present invention further relates to a method of diagnosing a neurological disorder, wherein the disorder is associated with Alzheimer's disease and/or wherein the disorder is associated with amyloid β (Aβ) pathology/amyloidosis in vitro comprising determining in a body fluid sample the presence of an anti-ankG autoantibody, wherein the presence or an elevated level of the antibody compared to the level in a control sample is indicative that an individual suffers from said disorder. Preferably the body fluid is cerebrospinal fluid or blood; see also Example 2, wherein significant occurrence of autoantibodies against ankG could be found in sera of AD in comparison to HCS patients. Furthermore cognitive decline over time was reduced in AD patients immunopositive against ankG.

Furthermore, the present invention provides a kit for use in the method for isolation an agent useful in the treatment of a neurological disorder or in the method of diagnosing a neurological disorder, as described above, said kit comprising ankG or a fragment thereof; APP or an ankG-binding fragment thereof; and/or an anti-ankG antibody.

In a further embodiment the present invention relates to an agent, in particular an ankG binding agent or an agent capable of interfering with the interaction of ankG and APP, or an agent capable of binding APP/ankG complexes, for use in in vivo imaging ankG or an ankG binding protein, or APP/ankG complexes, preferably wherein said agent is designed for in vivo imaging ankG or an ankG binding protein, or APP/ankG complexes, in the brain. Therefore, said agent may comprise a label (e.g., fluorescent, radioactive, enzyme, nuclear magnetic, heavy metal) and may be used as a probe to detect specific targets in vivo or in vitro including “immunochemistry” like assays in vitro. The specific label chosen may vary widely, depending upon the analytical technique to be used for analysis including detection of the probe per se and detection of the structural state of the probe. The label may be complexed or covalently bonded at or near the amino or carboxy end of the peptide. One example of indirect coupling is by use of a spacer moiety. In using radioisotopically conjugated peptides of the invention for, e.g., immunotherapy, certain isotopes may be more preferable than others depending on such factors as leukocyte distribution as well as stability and emission. Depending on the autoimmune response, some emitters may be preferable to others. In general, α and β particle emitting radioisotopes are preferred in immunotherapy. Preferred are short range, high energy a emitters such as ²¹²Bi. Examples of radioisotopes which can be bound to the peptides of the invention for therapeutic purposes are ¹²⁵I, ¹³¹I, ⁹⁰Y, ⁶⁷Cu, ²¹²Bi, agents which can be coupled to the peptides of the invention, as well as ex vivo and in vivo therapeutic protocols, are known, or can be easily ascertained, by those of ordinary skill in the art.

For example, peptide labeling with a metal isotope or a radioactive halogen isotope is described in international application WO 95/022341. In addition, international application WO 2004/013161 describes peptide aggregates that include assembling peptides optionally linked to metal binding moieties and/or target binding moieties as well as using such aggregates for magnetic resonance imaging.

In vivo near-infrared fluorescence imaging is also well known in the art; see, e.g., Frangioni, Current Opinion in Chemical Biology 7 (2003), 626-634. A review of imaging techniques such as ultrasound, CT (Computed Tomography), MRI (Magnetic Resonance Imaging), PET (Positron Emission Tomography), and molecular probes such as quantum dots and nanoshells and of their utility in system biology is given by Kherlopian et al., in BMC Systems Biology 2 (2008) 74 (DOI: 10.1186/1752-0509-2-74).

In in vivo embodiments, a labeled peptide or polpeptide, most preferably a probe comprising an labeled antibody is administered to a patient, such as by local injection, allowed to localize at any sites of target protein/peptide or higher order protein/peptide structures such as, e.g., heterogeneous extracellular insoluble protein-aggregates, such as β-amyloid plaques, present within the patient, and then the patient can be scanned to detect the sites of labeled probe localized at sites of target protein or higher order target protein structures. Other routes of administration also are contemplated, including intranasal and oral. As discussed above, the probe can be labeled with any label suitable for in vivo imaging. The patient can be subject to a full body scan to identify any site of target protein. Alternatively, specific areas of the patient can be scanned to determine whether target protein is localized in the specific areas. Specific areas of interest may include vascular tissue, lymph tissue or brain (including the hippocampus or frontal lobes), or other organs such as the heart, kidney, liver or lungs.

Accordingly, the present invention relates to in vivo imaging techniques employing any one of the peptides or polypeptides and in particular embodiments as well the antisense molecules of the present invention, hereinafter referred to as biomolecules. For example, the medical imaging technique Positron emission tomography (PET) which produces a three-dimensional image of body parts is based on the detection of radiation from the emission of positrons. Typically, a biomolecule is radioactively labeled, e.g. it incorporates a radioactive tracer isotope. Upon administration of the labeled biomolecule to the subject, typically by injection into the blood circulation, the radioactively labeled biomolecule becomes concentrated in tissues of interest. The subject is then placed in the imaging scanner, which detects the emission of positrons. In one embodiment, a labeled, preferably ⁶⁴Cu labeled agent of the present invention, preferably an agent capable of binding ankG is administered to a subject and detection of the agent is performed by placing the subject in an imaging scanner and detecting the emission of positrons, thereby indicating a neurological disorder if for example emission within plaque-resembling structures is detected. The present invention thus encompasses a method for PET imagining, comprising the step of administering a ⁶⁴Cu-labelled or equivalent labeled agent of the present invention to a subject.

As noted above, in some embodiments, the probe is modified to comprise labels that are detectable by optical means. Such labels may include tryptophan (an amino acid), pyrene or similar fluorophores, or a fluorescent protein, attached at or near the terminal positions of the peptide probe. Attachment of labels such as fluorophores is achieved according to conventional methods which are well known in the art. Another class of fluorescent probes called quantum dots (QD) may be used as well, which are a class of polymer-encapsulated and bioconjugated probes that can fluoresce at multiple wavelengths spanning the visible spectrum; see, e.g., for review Mansour and Kazmierczak, Clinical Biochemistry 40 (2007), 917-927.

In a further embodiment the present invention relates to a method of reducing brain Aβ pathology and lowering brain levels of Aβ comprising administering to a subject in need thereof a therapeutically effective amount of an agent as defined herein and above. For example, the agent used in said method of reducing brain Aβ pathology and lowering brain levels of Aβ according to the present invention may be any agent as defined herein and above capable of inducing an immune response in a subject, e.g., recombinant ankG protein or a fragment, derivative or analog thereof introduced in the way of vaccination as shown in Example 3. Analogical, any agent capable of binding to ankG may be used in said method, e.g., by passive immunization with antibodies against ankG as shown in Example 4. A further, not limiting example of an agent used in said method may by any agent as defined herein and above capable of lowering the expression of ankG, e.g., an antisense oligonucleotide as shown in Example 6.

Hence, the present invention generally relates to the use of agents of the present invention for the preparation of a composition or kit for the prevention, amelioreation, treatment or diagnosis of a disease, monitoring of the progression or therapy of a disease, in vitro or in vivo studies aiming at elucidation of the mechanisms underlying a disease, screening of ankG binding compounds, preferably antibodies, or screening for drugs, preferably drugs interfering with extracellular localization of ankG, or interfering with the binding of ankG to a target protein in the brain. Preferably, said disease is associated with Alzheimer's disease. In one embodiment of the present invention, the use involves the detection of said protein, or aggregates comprising said protein, for example by MRI, NIR or PET.

For example, in the context of AD, ankG binding agents/probes can be used to identify exosomes containing ankG, extracellular ankG or aggregates thereof, or App/AnkG complexes or aggregates comprising such complexes, present in a sample. For example, the presence and load of ankG, extracellular aggregates containing ankG and ankG autoantibodies, or App/AnkG complexes or aggregates comprising such complexes, can be used to identify a patient at risk for AD or a patient suffering from AD, and/or the extent to which the disease has progressed. The same information also could be used to determine the need for a therapeutic regimen or for a more or less aggressive regimen than currently being used, and to monitor the efficacy of a given therapeutic regimen.

In one embodiment, ankG binding agents/probes are used to determine the localization of ankG or higher order structures containing ankG, i.e. ankG comprising extracellular protein aggregates or plaques within the patient. For example, biological samples from specific segments of the brain can be obtained and analyzed for the presence of ankG, exosomes or amyloid plaques containing ankG. Alternatively, labeled probes can be administered to the patient, such as by local injection, allowed to localize at any sites of ankG or extracellular aggregates comprising ankG present within the patient, and then the patient can be scanned to detect the sites of labeled probe localized at sites of ankG or β-amyloid plaques comprising ankG. Specific sites of interest might include the hippocampus or cortical brain areas. Other sites of interest might include vascular tissue, lymph tissue, and other organs such as the heart, kidney, liver or lungs.

General methods in molecular and cellular biochemistry useful for diagnostic purposes can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996). Reagents, detection means and kits for diagnostic purposes are available from commercial vendors such as Pharmacia Diagnostics, Amersham, BioRad, Stratagene, Invitrogen, and Sigma-Aldrich as well as from the sources given any one of the references cited herein, in particular patent literature.

The present invention also relates to a kit for use in any one of the methods as described above, i.e. for identifying, isolating, determining and/or using the polypeptide and polypeptide aggregates of the present invention, said kits containing specific reagents such as those described hereinbefore, for example a peptide, polypeptide or fusion protein of the present invention, a recombinant nucleic acid molecule encoding said peptide, polypeptide or fusion protein, an expression vector comprising said nucleic acid molecule, which is operatively linked to an expression control sequence, a host cell comprising said nucleic acid molecule or expression vector, preferably the host cell is E. coli; and optionally a protease, preferably TEV or a corresponding expression host; see also the Examples. The kit may further comprise for example selectable markers, reference samples, microarrays, culture vessels, and maybe some monitoring means. The kit preferably comprises at least one recombinant polypeptide of the present invention, preferably ankG protein or fragment thereof, as well as reference molecules for indicating the potential drug efficacy of an added agent, wherein the reagents are preferably kept in single containers. The kit of the present invention is preferably suitable for commercial manufacture and scale and can still further include appropriate standards, positive and negative controls. It preferably further comprises at least one reagent which is selected from the group consisting of reagents that selectively detect the presence or absence of ankG, for example an anti-ankG antibody.

Such kit would further typically comprise a compartmentalized carrier suitable to hold in close confinement at least one container and the compounds of the kit may be sterile, where appropriate. The kit may further include a transfer means, such as pipes for transferring the reagents or cells. In other embodiments, there may be components for application of agents, compounds or compositions to an individual, preferably an animal, such as a syringe, a needle, and so forth. The kit may further comprise components for extracting for example cells from a tissue of interest. Furthermore, instructions can be provided to detail the use of the components of the kit, such as written instructions, video presentations, or instructions in a format that can be opened on a computer, e.g. a diskette or CD-ROM disk. These instructions indicate, for example, how to use the cell, agent, compound, composition and the like to screen test agents of interest. Most preferably, the instructions refer to the use of the kits in the methods concerning the identification and/or isolation of interacting molecules of ankG or validation or assessment of potential drugs, agents, compositions or compounds influencing, either inhibiting or enhancing said interaction.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness are given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

The Examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed. by Beers and Berkow (Merck & Co., Inc., 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251.

### Material and Methods

### Human Samples:

AD patients and HCS were recruited at the gerontopsychiatric department of the University of Zurich. AD cases used for sera analysis had an age at onset ≧65 years and a diagnosis of definite or probable AD according to the National Institute of Neurological and Communication Disorders and Stroke-Alzheimer's Disease and Related Disorders Association diagnostic criteria. HCS subjects were matched for age and sex. Cognitive assessment was recorded using MMSE. All subjects were followed up at yearly intervals for a period of up to 26 months with repeat MMSE on each occasion. The rate of cognitive decline for each group was based on the average slope of MMSE weighted for numbers of months of follow up. Human brains were obtained from the Kathleen Price Bryan Brain Bank from Duke University (USA). AD classification was assessed according to (52, 53).

### Tissue Micro Arrays (TMA):

Immunohistochemical quantification of ankG expression was performed by TMA analysis from 288 tissue samples, each from AD and HCS (n=12). The selection of specimen and TMA construction were previously described (22). Primary antibodies were visualized using either a standard diaminobenzidine horseradish-peroxidase method for Aβ (Histofine simple Stain Max PO, Nichirei Bioscience, Nichirei, Japan) or alkaline phosphatase method for ankG staining (Histofine simple Stain Max AP, Nichirei). AnkG staining was assessed in 7 grades determined by the sum of a score for the % of immunopositive cells (0=none, 1=1-10%, 2=10-50%, 3=>50%) and the intensity of the staining (0=none, 1=weak, 2=moderate, 3=strong, 4=very strong).

### Animals.:

ArcAbeta mice were produced as described previously by (17). Tg2576 mice were bred from germinal lines produced as described previously (27). Non-transgenic littermates and APP deficient mice were used as controls when specified. All animals were housed 3-5 per cage and had access to food and water ad libitum under a 12-hour light/dark cycle. All in vivo experiments were performed with the approval of the Swiss veterinary council (BVET).

### Anaesthesia and Perfusion.:

Mice were anaesthetized using a cocktail of 1.25 ketamine and 0.25% xylazine. After transcardial perfusion with ice-cold buffer PBS (10 mM Na₂HPO₄, 2.5 mM NaH₂PO₄, 150 mM NaCl, 3 mM KCl) one hemisphere was processed biochemically and the other one incubated in paraformaldehyde (PFA) 4% in PBS for 4 hours at 4° C. followed by incubation in sucrose 30% in PBS for 24-36 h for immunohistochemistry purposes.

Active ankG immunization.:

ArcAbeta mice or Tg2576 mice and their non-transgenic littermates were actively immunized with either 10 μg ankG protein in PBS+Freund's adjuvant complete (FA) (Sigma Aldrich F5881) or PBS+FA (control-immunized). Blood was drawn from the tail of each mouse before and during (every 4 weeks) immunization to obtain sera for antibody titre analysis. Immunization cocktails were injected subcutaneously between the scapulae. Three 4-weekly immunization re-boosts were performed with Freund's adjuvant incomplete instead of FA. At the end of the 3-months immunization period the mice were perfused. One hemisphere of the brain was fixed in paraformaldehyde solution for 4 hours at 4° C. and subsequently put in a solution of 30% sucrose at 4° C. for cryoprotection for 24-36 hours for immunohistochemistry. The other hemisphere of the brain was processed for biochemical analysis.

### Neurological Examination.:

Mini neurological examination was performed before, during and after each immunization as described in (17). Brief, all animals had normal fur appearance, no secretory signs, normal body postures and normal basic reflex, including the eye blink, pupillary, flexion and righting reflexes. All animals had age-appropriate body weight and no difference in muscular strength (grip strength) as measured with a spring scale.

### Cell Lines.:

Human embryonic kidney cells (HEK 293, DSMZ, ACC 305) were cultured in Dulbecco's modified eagle medium (DMEM, Invitrogen #52100039) supplemented with 10% fetal calf serum (FCS) and penicillin/streptomycin (PenStrep, Invitrogen #10378-016) at 37° C., 5% CO₂, 95% humidity. SHY-5Y neuroblastoma cells were grown in DMEM/F12 supplemented with 20% fetal bovine serum. For differentiation, cells were sequentially treated for 5 days with 5 μM retinoic acid (Sigma, Buchs, Switzerland, Cat. No. 68268) and for 5 days with 20 ng/ml BDNF (Peprotech, London, UK, Cat. No. 450-02-10 ug), with serum reduced to 2%, after which they were transfected identically to HEK293 cells. HEK293 cells expressing APP-Citrine were obtained and cultured as described in (54). HeLa cells stably expressing Swedish APP.

siRNA Silencing.:

Silencing of the ankG gene (Validated siRNA cat. no. SIO2780204, Qiagen AG, Switzerland), ankB gene (Validated siRNA cat. no. SI03031238, Qiagen AG, Switzerland) and negative control siRNA Alexa Fluor 488 (cat. no. 1022563), Qiagen AG, Switzerland) in differentiated SHY-5Y cell, HEK293, Citrine-HEK293, Swedish APP HEK293 and Swedish APP HeLa cells was carried out using the RNAi kit and RNAi nucleotides as per manufacturer's instructions (Qiagen). Silencing was repeated every 24 hours for 3 days followed by immunoblotting analysis. To inhibit γ-secretase activity, cells were cultured in the presence of 1 nM DAPT (Sigma Aldrich, Switzerland, Cat. No. 208255-80-5) as described in (54).

### Protein Antigen Arrays Screening.:

The protein antigen arrays screening was custom-made by imaGenes GmbH, Berlin, Germany, containing 37,000 clones from a human fetal brain cDNA expression library as described in (22). Briefly, the protein arrays were utilized for high-throughput antibody screening and each clone was spotted in duplicates onto PVDF filter membranes (2 parts, each 26 cm×26 cm in size) (Fischer Scientific, Cat. No. IPFL00010). For screening with sera antibodies, the protein array filters were first incubated with blocking buffer (3% non-fat milk in Tris-buffered saline [TBS]) for 2 hours and then incubated with the patient's sera (dilution 1:1000) for 14 hours. After extensive washing with TBS containing 0.05% Tween-20 and 0.5% Triton X-100 (TBS-T), filters were incubated with HRP-conjugated anti-human IgG (1:2000 diluted in blocking buffer) (Abcam, UK, Cat. No. ab 87422) for 1 hour. After several washings, the filters were developed by stabilized tetramethylbenzidine-blotting (TMB-blotting) substrate (Pierce, USA, Cat. No. 34021) for 5 minutes. Positive spots in duplicates indicated specific binding of sera antibodies to recombinant proteins. The corresponding expression clones were obtained from the RZPD and were cultured in LB Broth Base medium (Invitrogen Corp., Carlsbad, USA, Cat. No. 12780-029) supplemented with ampicillin (Teknova, Hollister, USA, and Cat. No. A9510). Their plasmids were isolated, and the cDNA inserts were sequenced for identification of the proteins. Immunoreactivity was assigned with the following score: (0=none, 1=weak, 2 moderate, 3=strong, 4=very strong). The strongest immunoreactive clones encoding for proteins expressed in the correct reading frame and which were immunopositive in at least 2 different sera were considered as positive hits.

### Image Analysis.:

Immunohistochemical images were acquired on a Leica DM4000B microscope using an Olympus DP71 colour digital camera and newCAST software (Visiopharm, Copenhagen, Denmark). Image analysis was carried out with ImageJ image analysis software (56, 57). Quantification of the number of β-amyloid plaques and Iba-1 positive microglia was performed using the area measurement tool. For measurement of plaque diameter the particle count tool was used. The measurements were done on 10 sequential sections per mouse.

WB analysis.:

Densitometric analyses of the WBs were done using ImageJ (Scion Corporation, Frederick, Md., US), (56, 57) or Tina Image analysis software (University of Manchester, UK; http://tina-vision.net). Mean optical intensities were plotted after standardization of intensities to loading controls.

### Statistical Analysis.:

Data analysis, statistical evaluation and graphical representation of data were done using

GraphPad Prism software (GraphPad Software, La Jolla, USA, http://www.graphpad.com/prism/Prism.htm). Mann Whitney test, t-test or Chi-square test was used to determine significance between the groups.

### Biochemical Enrichment of Aβ.:

Frozen brain samples were homogenized in 1% TritonX-100 in buffer A (50 mM Tris-Cl, 100 mM NaCl, pH 8). The homogenate was centrifuged at 100000 g for 1 hour. The pellet obtained was resuspended in 2% SDS and 2 mM EDTA in buffer A. The pellet obtained after centrifugation at 100000 g for 1 hour was enriched in Aβ. The protein concentration of each fraction was quantified by BCA assay kit (Pierce, Rockford, Ill., USA Cat. No. 23225) and subjected to immunoblotting and Aβ ELISA. All buffers were used at 4° C. with protease inhibitors (Roche Applied Science, Indianapolis, Ind., USA, Cat. No. 11873580001).

### Exosome Preparation:

Exosomes were isolated from HEK 293 cells. Briefly, cells from 3 to 6 T175 flasks (Sigma Aldrich, St. Louis, USA, Cat. No. CLS 3298) were cultured in DMEM with 10% FCS (Gibco, Invitrogen Corp., USA, Cat. No. 10108-157). A day before the exosome preparation, culture medium was replaced with AIM-V medium (Gibco, Invitrogen Corp., USA, Cat. No. 1205509). Culture supernatants of cells grown for 24 hrs in AIM-V medium were collected and spun at 300×g for 10 min to remove cells. The supernatants were then sequentially centrifuged at 1200×g for 20 mins, 10,000×g for 30 mins, and 100,000×g for 1 hour. The 100,000×g pellet was washed with Phosphate buffer saline (PBS) and again spun at 100,000 g for 1 hour. The second 100,000×g pellet (exosomal pellet) was resuspended in PBS. The pelleted fractions were then used for immunoblotting.

### Immunoblotting.:

Equal amounts of total protein were subjected to separation on 10-20% Tricine gels (Invitrogen, Basel, Switzerland), blotted on nitrocellulose membranes (0.45 μm; Biorad, Reinach, Switzerland, Cat. No. 162-0116) or Protran BA 79 cellulose-nitrate membranes (0.1 μm; Schleicher & Schuell, Dassel, Germany (Cat. No. 732-4018). The immunoblot was then incubated with primary antibodies (table S3) followed by incubation with HRP-tagged secondary antibodies.

Detection was performed using chemiluminescence visualized using ECL WB reagents (GE, Germany, Cat. No. RPN2109) or SuperSignal West Dura Extended Duration reagents (Pierce, Rockford, Ill., USA, Cat. No. 34075) on BIOMAX films (GE, Germany, Cat. No. 25010141).

TABLE S3
Antibodies and their application

| | | | | Antigen |
|---|---|---|---|---|
| Antigen | Company | Product# | Application(s) | retrieval |
| ankG | Santa Cruz | sc-28561 | WB, IP | NA |
| | Santa Cruz | sc-31778 | ELISA | NA |
| | Zymed ® Laboratories | 33-8800 | IHC | HCl |
| | In-house produced monoclonal | NA | IHC | Protein K |
| ankB | Zymed ® Laboratories | 33-3700 | WB | NA |
| APP C-term. | Sigma | A8717 | WB | NA |
| APP N-term. | Millipore | MAB348 | WB | NA |
| APP/Aβ | Covance/Signet | SIG-39300 | WB, IHC, IP | HCl: IHC, NA: |
| | | | | WB & IP |
| | Dako | M0872 | IHC | NA |
| Actin | Abcam | ab6276 | WB | NA |
| Tau | Innogenetics | clone HT7 | WB | NA |
| Neurofilament | Millipore | AB1982 | IHC | HCl |
| 200 kDa | | | | |
| Caspr P6061 | Gift from Dr. E. Peles | | | |
| TAP1 | (51) | | | |
| Alix | Biolegend | 634501 | WB | NA |
| Calnexin | Stressgen | SPA-865 | | |
| Iba 1 | Wako | 1919741 | IHC | NA |

IHC = immunohistochemistry,
IP = immunoprecipitation,
WB = WB,
NA = not applicable

### Immunoprecipitation.:

Brain homogenates were lysed for 1 hour with lysis buffer, pH 7.5, containing 50 mM Tris-HCl (Sigma Aldrich, Cat. No. T5941), 150 mM NaCl (Sigma Aldrich, Cat. No. S5886), 1% Nonidet P-40 (Sigma Aldrich, Cat. No. 74385), 1 mM Na₂P₂O₇ (Sigma Aldrich, Cat. No. 221368), 1 mM NaF (Sigma Aldrich, Cat. No. 47072), 2 mM Na₃VO₄ (Sigma Aldrich, Cat. No. S6508), 0.1 mM PMSF (Sigma Aldrich, Cat. No. 7626), 2 mM EDTA (Sigma Aldrich, Cat. No. EDS-100G) and EDTA-free protease inhibitor cocktail (Roche, Roche Applied Science, Indianapolis, Ind., USA, Cat. No. 11873580001). Lysates were precleared with non-specific IgG antibodies followed by protein A/G coated magnabeads (=Dynabeads® Invitrogen, USA, Cat. Nos. 100-01D, 100-03D), for 1 hour each. The precleared lysate was then incubated with indicated primary antibodies or non-specific IgG for 1 hour. Antibody complexes were then precipitated with beads applied for 1 hour. Beads were washed 5 times with lysis buffer and once with PBS and used for WB immunoblot analysis. All steps were carried out at 4° C. For every 1 mg/ml of total protein content of homogenate 3 μg antibodies were used

### Serum Antibody Titres.:

Blood samples were spun down at 3000 g for 10 minutes at 4° C. to obtain sera for titre analysis. Anti-ankG antibodies in the serum of immunized mice were determined by ELISA techniques. Briefly, 0.5 μg/ml rat recombinant ankG (or 2 μg/ml BSA) in PBS were immobilized on the polyvinylchloride surface overnight at 4° C. Wells were washed five times for 5 minutes at room temperature (RT) with PBS containing 0.05% Tween-20 (PBS-T) followed by blocking for 1 hour at RT with 2% BSA in PBS and subsequently incubation with immunized sera diluted 1:500 in PBS-T containing 3% BSA for 2 hours at RT. Wells were washed and bound serum antibodies were detected with HRP-conjugated anti-mouse antibodies. The reaction was developed with 0.1% ABTS (Roche) in 100 mM acetate buffer, pH 5.0. The reaction was stopped with 100 mM sodium fluoride. OD was measured at 450 nm.

### Protein Ligand Binding Assay.:

APP CTF-50 peptide (Calbiochem, EMD Chemicals, USA, Cat. No. 171545), Aβ40 and Aβ42 (Bachem AG, Weil am Rhein, Germany, Cat. Nos. H-1194, H1368) were coated on 96-well polyvinyl chloride plates at 0.5 μg/ml in PBS at 4° C. overnight. Wells were then blocked for 1.5 hours with PBS containing 2% BSA and incubated for 2 hours at RT with rat recombinant ankG (0-2.5 μg/ml) diluted in PBS-T. Washing steps were carried out between all incubations using PBS-T. Plates were incubated for 1.5 hours at RT with goat antibodies against ankG in PBS-T containing 2% BSA. Detection was as described before for serum titre analysis.

ELISA Quantification of Aβ levels.:

Concentrations of Aβ40 and A042 from mice serum and SDS insoluble brain fractions were quantified using commercially available ELISA kits (Millipore, Billerica, USA, Cat. Nos. EZHS40, EZHS42) as per manufacturer's instructions. Sera were diluted 1:100 in PBS before use. Two to 4 μg at 25 μg/ml of total protein of SDS insoluble fractions in PBS was used per well.

### Immunohistochemistry.:

Fixed and cryoprotected hemibrains were cut in 30 μm thick slices at ˜−80° C. using a microtome (Leica Jung HN40) and kept at −20° C. in an anti-freeze solution (phosphate buffer 0.50 M in MilliQ water:ethyleneglycol:glycerol=1.3:1:1) until staining was performed. All immunohistochemical stainings were executed using the free-floating method. Washing steps were carried out between all incubations using washing buffer (TBS pH 7.4 containing 0.2% Triton X-100 (Sigma-Aldrich, Cat. No. X100-5ML) at RT. Antigen retrieval was performed when required (Table S3) by incubating the slices in 1 M HCl at 65° C. for 30 minutes. Slices were blocked for 1 hours at RT using blocking buffer (5.0% goat serum 5.0% donkey serum in washing buffer). Blocked slices were incubated overnight at 4° C. with slight agitation in primary antibody incubation buffer (2.5% goat serum and 2.5% donkey serum in washing buffer). This was followed by secondary antibody incubations were carried out for 2 hours at RT. Slices were washed in washing buffer, mounted on chrom-gelatin-coated microscopy slides (Super-frost-plus, Menzel, Braunschweig, Germany, Cat. No. J1800AMNZ) and glass-covered using Hydromount® (National Diagnostics, Hull, UK, Cat. No. HS-106).

### Organotypic Hippocampal Slice Cultures and Sindbis Virus Infection:

Organotypic hippocampal slice cultures were prepared and cultured according to (55). Briefly, six to seven day old arcAbeta transgenic and non-transgenic mice were decapitated, brains were removed, both hippocampi isolated and cut into 400 μm thick slices using tissue chopper. Slices were cultured on Millicell culture plate inserts (0.4 μm, Millipore, Bedford, Mass., Cat. No PF187EN00) in six well plates containing 1 ml culture medium (46% minimum essential medium eagle with HEPES modification, 25% basal medium with earls modification, 25% heat-inactivated horse serum, 2 mM glutamine, 0.6% glucose, pH7.2). Culture plates were kept at 37° C. in a humidified atmosphere containing 5% CO₂. Slices were kept in culture for 12 days before the experiments. Culture medium was exchanged every second to third day. On day 11 culture medium was replaced by low-serum Nb-N1 medium (94.5% Neurobasal medium, 0.5% heat-inactivated horse serum, 2 mM glutamine, 0.6% glucose, 1× N1 supplement, pH 7.2). On day 12 in vitro slice cultures were infected with Sindbis virus expressing EGFP using droplet method (29). For spine analysis, cultures were fixed at day 3 post infection within six-well plates. Slices were left attached to the culture plate membrane to preserve hippocampal structure and rinsed with PBS. Slices were then fixed with 4% paraformaldehyde in PBS containing 4% sucrose for 2 h at 4° C. After washing with PBS, cultures were mounted and coverslipped. For analysis of effects of anti-ankG antibodies cultures were treated with 10 μl antibody per 1 ml culture medium for 1 week.

### Confocal Imaging of Fixed Hippocampal Slice Cultures and Analysis of Spine Density:

Confocal high-resolution imaging of spines was performed using TCS/SP2 Leica confocal laser scanning microscope with 63× objective (oil, NA: 1.4). Fragments of apical and basal dendrites of hippocampal CA1 and CA3 pyramidal neurons were imaged with voxel size of 0.058×0.058×0.25 μm in x-y-z direction. To determine spine density the length of the dendrite was measured and spines were counted as protrusions in x- and y-axes using NIH ImageJ software (56, 57).

### Example 1

### The Cytoskeletal Adaptor Protein ankG is Redistributed into β-Amyloid Plaques in AD Brains

To characterize the role of ankG in AD neuropathology, the distribution of ankG was studied in brains. Immunohistochemistry of AD hippocampal tissue revealed the presence of ankG in β-amyloid plaques (FIG. 1a). As expected (16), ankG was also present in neuronal cell bodies and axons in both AD and HCS brains. Interestingly, a similar pattern of ankG immunostaining was present in β-amyloid plaques and neurons of cortical sections from a transgenic mouse model for AD (FIG. 1b). This mouse model expresses human APP with the Swedish and Arctic familial AD-causing mutations (arcAbeta mice) (17). Expression of ankG and APP in dystrophic neurons was increased in older mice (FIG. 1b, lower panel) as compared to younger animals (FIG. 1b, upper panel), a finding correlating with disease progression. Immunohistochemical quantification of ankG in human frontal cortex showed an increase in ankG expression in AD versus HCS (FIG. 1c). AnkG co-distributed together with tau and Aβ on WB of SDS-insoluble protein fractions from human frontal cortex samples and increased levels of ankG in AD versus HCS were observed (FIGS. 2a, 2b). As expected, tau protein levels were also increased (FIG. 2a) (18). Similar to human brain, ankG distribution was affected in hippocampal sections from 24 months old arcAbeta mice as compared to their non-transgenic littermates (FIG. 2c). In contrast, the staining patterns of neurofilament—a neuronal marker—showed intact gross hippocampal architecture (FIG. 2c). The redistribution of ankG into β-amyloid plaques also occurred in brains of 7 months old arcAbeta mice. These results suggest that redistribution of ankG from the neuronal cytoplasm to β-amyloid plaques could be due to a pathogenic mechanism occurring in AD rather than to a general effect of neurodegeneration or aging on brain architecture. It is known that cytosolic proteins such as heat shock proteins, PrP, Aβ and caspase-1 are released from the cell through exosomes which are small membrane vesicles involved in trafficking, cell-cell communication and immune response activation (19, 20). AnkG was found in exosomes purified from HEK293 cells (FIG. 2d), suggesting an exosome-mediated extracellular ankG release.

### Example 2

### AnkG is Associated with an Immune Response in AD

The abnormal extracellular and increased ankG expression pattern observed in AD brain and the presence of ankG in exosomes lead the inventors to hypothesize that ankG could provoke an immunoresponse in AD patients. To explore this possibility Western blot (WB) analyses was performed using purified ankG probed with sera from 14 AD patients and 14 HCS (table 51) (FIGS. 3a, 3b).

TABLE S1
Age and MMSE of the patient population

| | | MMSE |
|---|---|---|
| | Age average | average |
| AD | 74 ± 4.6 | 19.3 ± 6.1 |
| HCS | 70.4 ± 7.8 | 29.7 ± 0.4 |

Eight AD patients and only 2 of the HCS showed IgG against ankG (FIG. 1b). No IgM against ankG were found in these sera. The same AD sera did not cross-react with purified ankyrin B (ankB), a highly homologous protein of ankG (21) confirming that ankG antibodies found in AD patients were not the result of a general unspecific immunogenic reaction against members of the ankyrin family. The rate of cognitive decline over time in the AD population (n=12 out of 14; 2 patients had only one cognitive assessment) was also assessed. Linear regression analysis of decline in the mini-mental score evaluation (MMSE) weighed for the number of follow-up months showed a significant reduced rate of cognitive decline in ankG-immunopositive versus immunonegative patients (table S2).

The limitation of this observation, however, is the population size and hence future studies with larger independent populations are required. To further prove the existence and specificity of antibodies against ankG in AD sera protein arrays generated from a human fetal brain cDNA expression library comprising 37,000 clones (22-24) were used and probed with an independent set of AD sera. In addition to ubiquitously expressed genes, this library contained a subset of proteins that are predominantly expressed in brain tissue. Sera from 15 AD patients and 10 age-matched healthy control subjects (HCS) were applied to separate protein arrays (FIG. 3c). A maximum of 15 clones were identified in each subject. Sequencing of immunopositive clones identified ankG among these proteins. These results suggest that ankG provokes an immunoresponse in AD patients with detectable antibodies in the sera

TABLE S2
Comparison of cognitive score changes over time versus baseline in AD
patients either immunopositive or immunonegative for sera antibodies
against ankG

| | | AnkG | |
|---|---|---|---|
| | AnkG | immunopositive | |
| | immunonegative | AD patients | |
| | AD patients (n = 6) | (n = 6) | P value |
| MMSE score at | 23.5 ± 1.5 | 17.5 ± 6.55 | |
| baseline ± SD | | | |
| Slope (points per | 0.40 ± 0.37 | 0.13 ± 0.29 | 0.04 |
| each 6 months) ± SD | | | |

### Example 3

### Active Immunization with ankG Reduces β-Amyloid Pathology In Vivo

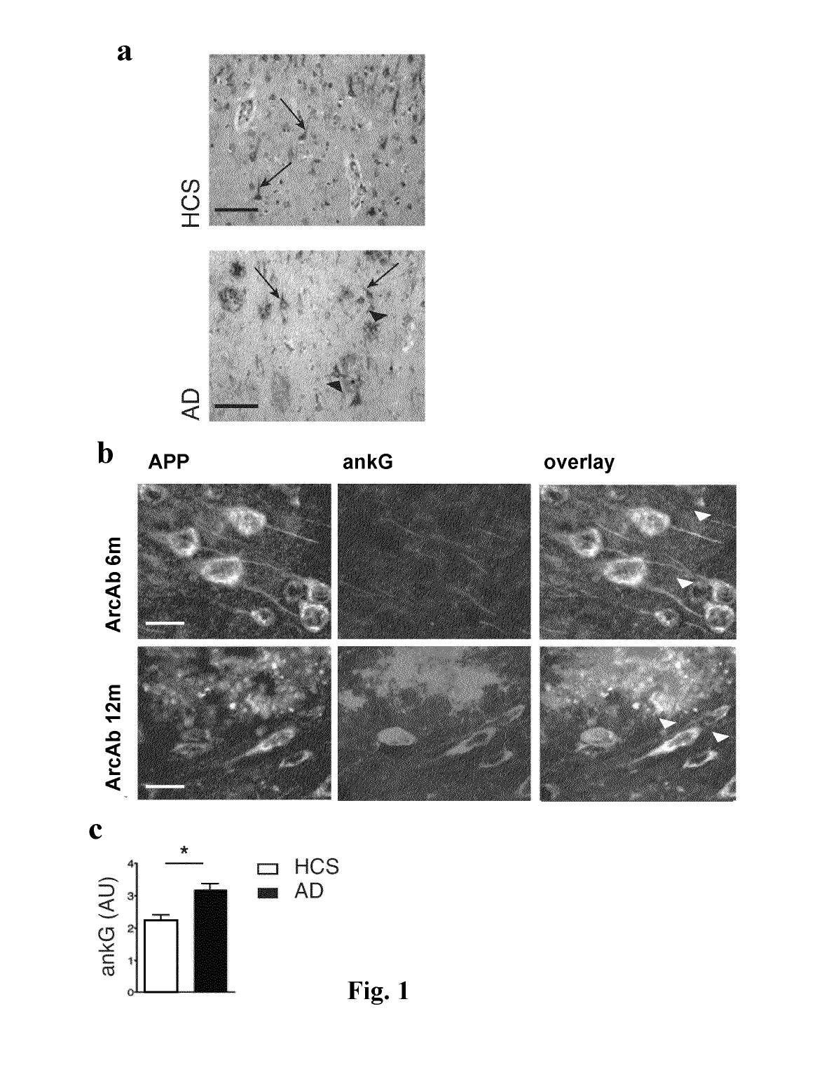

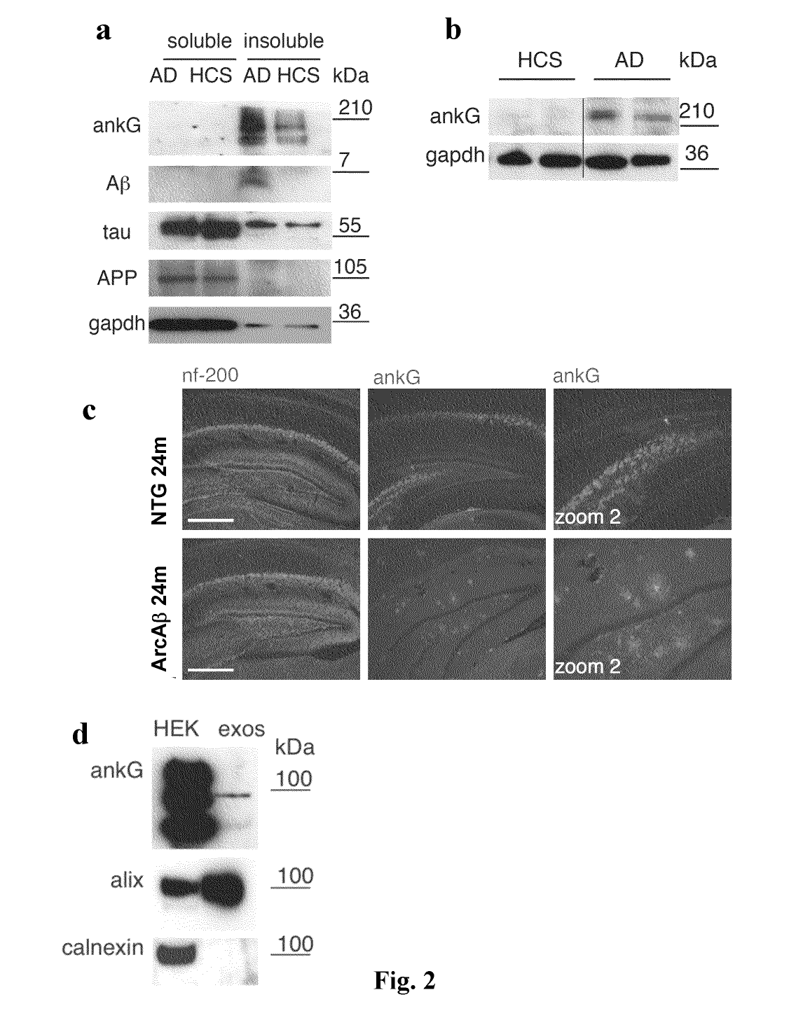

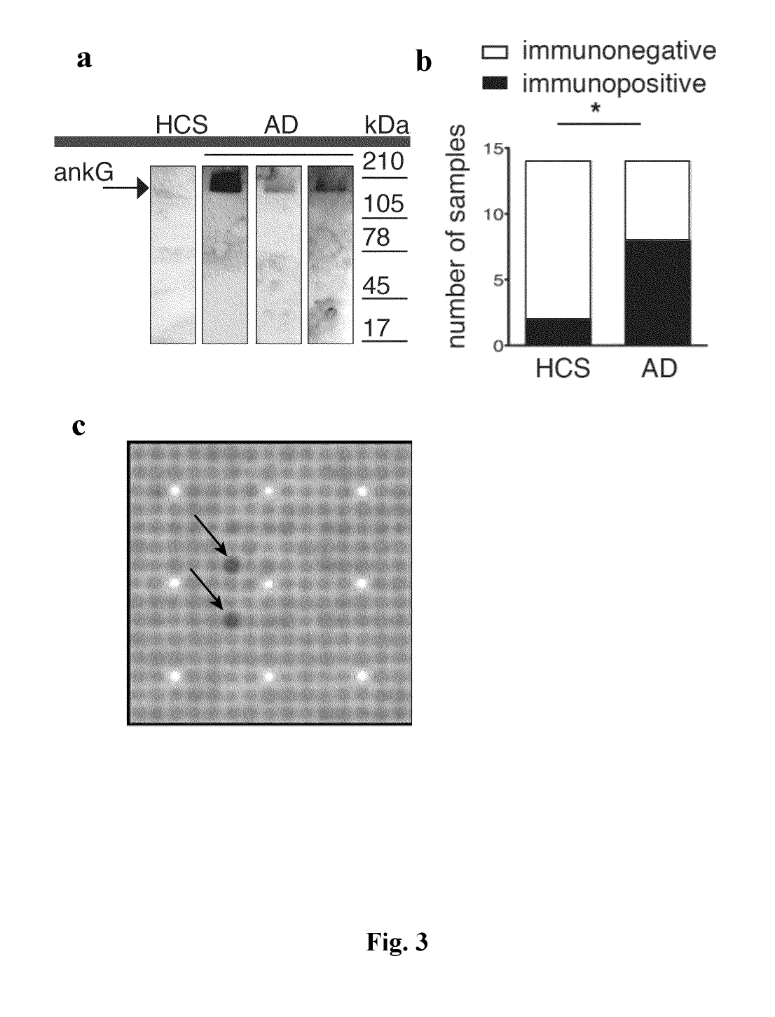

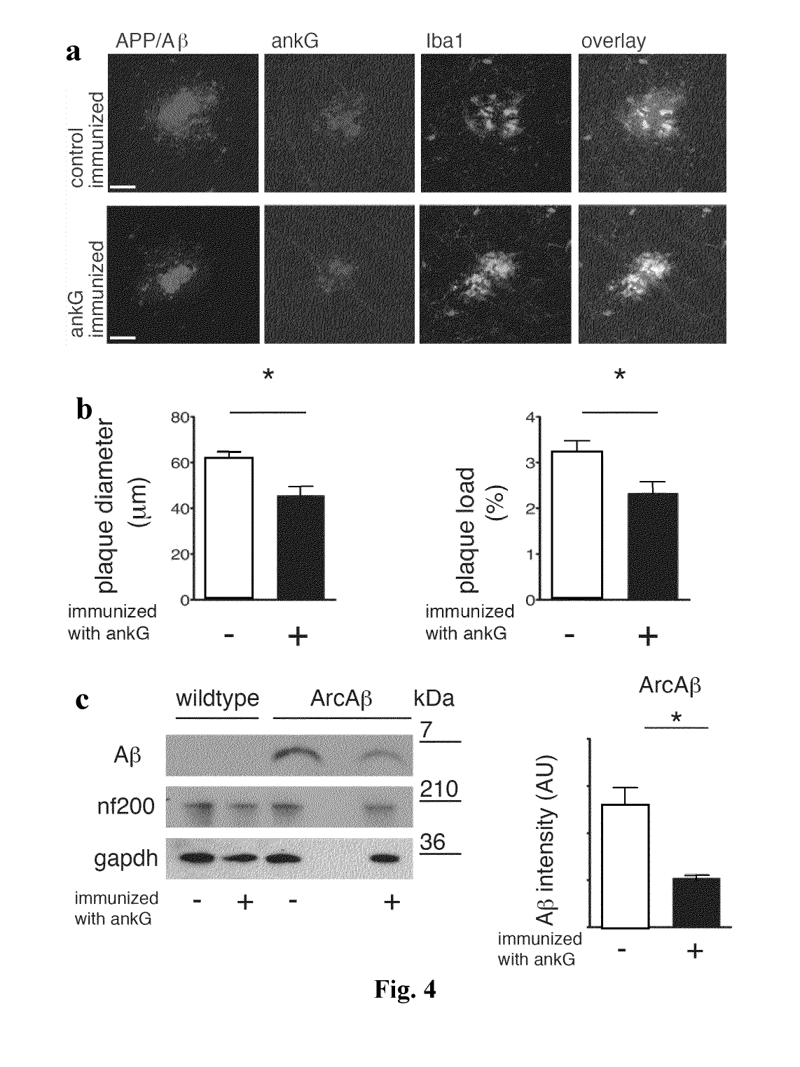

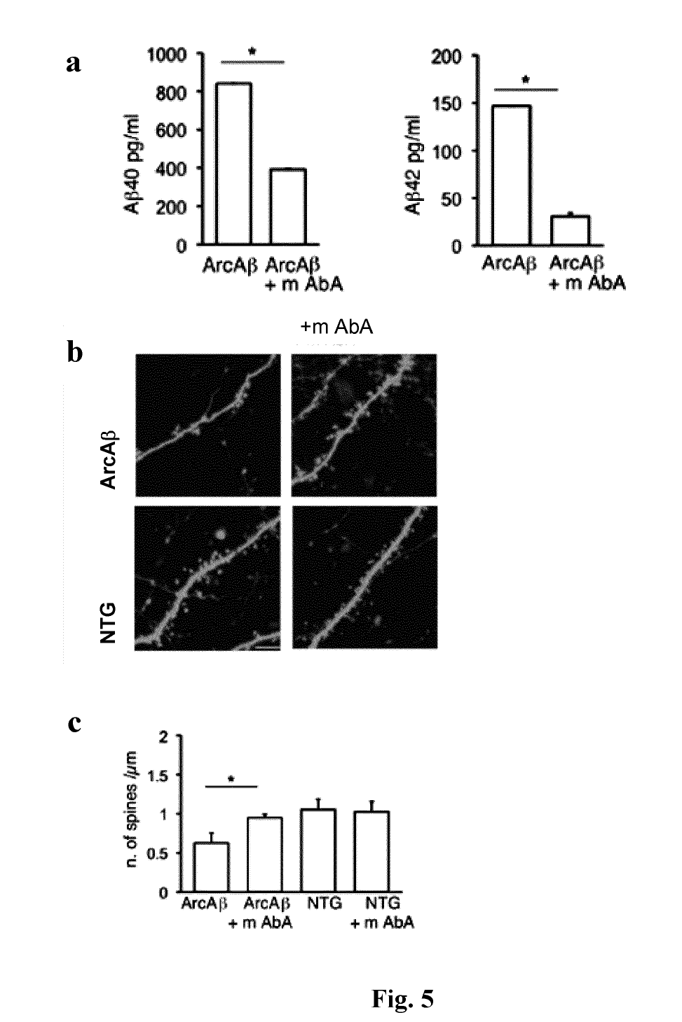

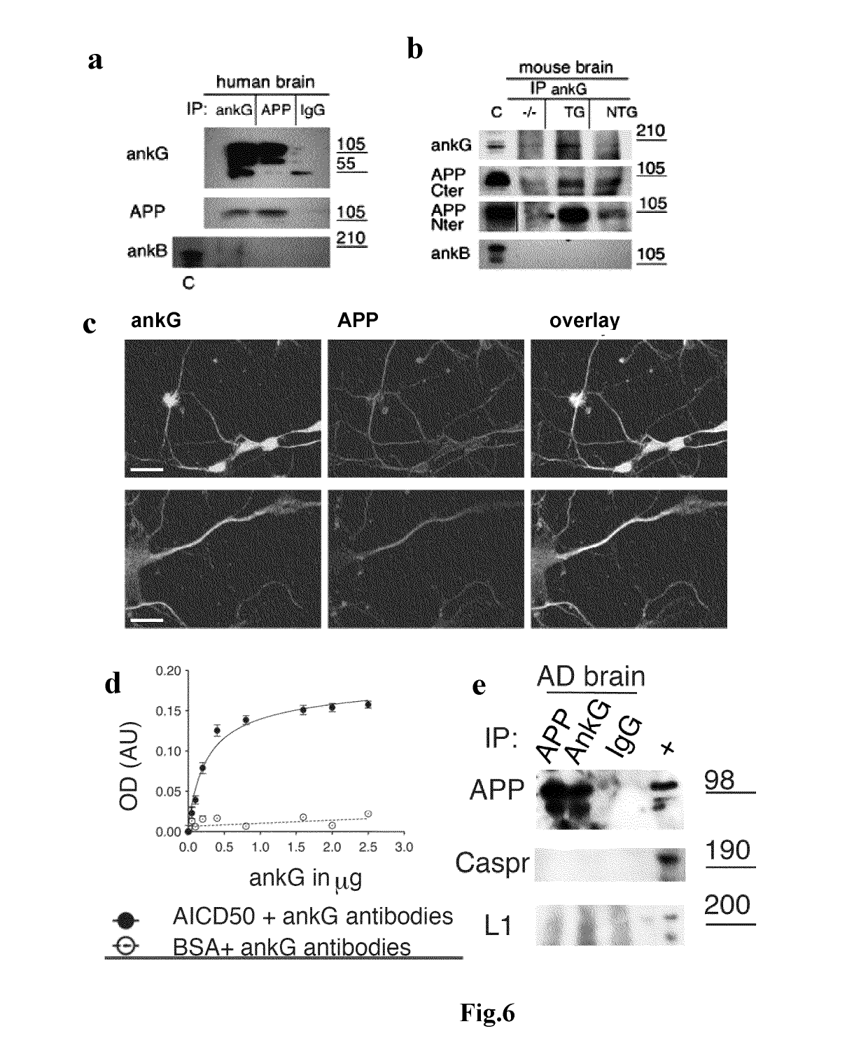

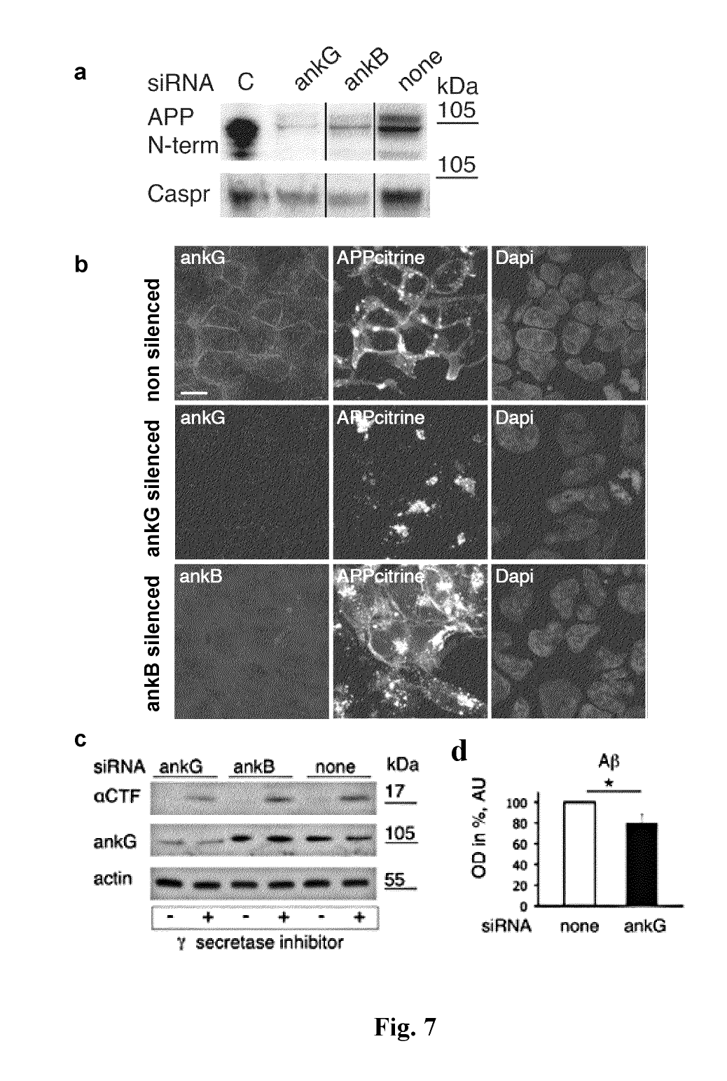

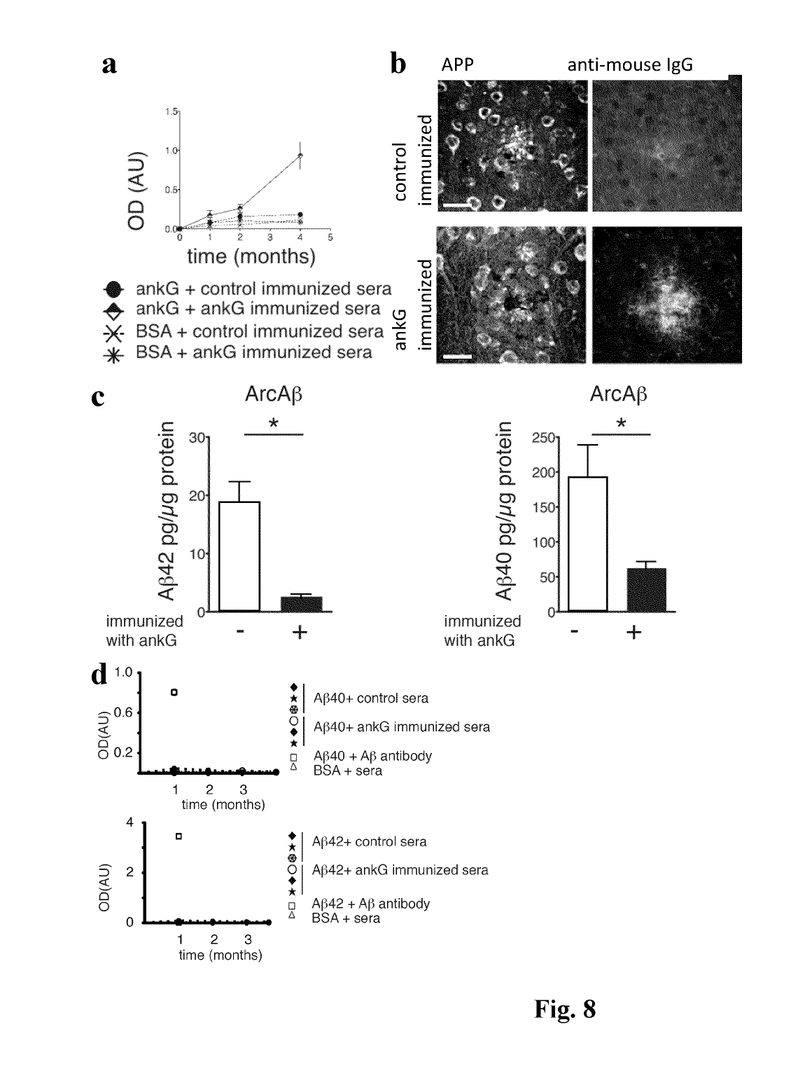

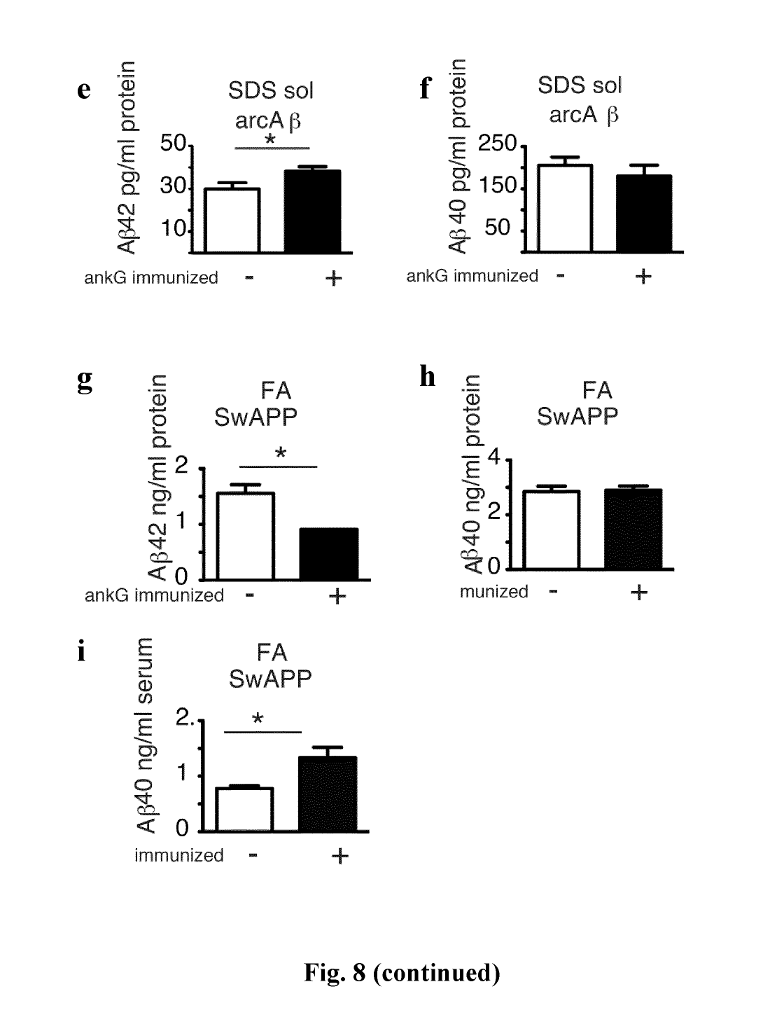

To study whether targeting ankG by immunotherapy affects the clearance of (3-amyloid and to experimentally mimic the immune response observed in humans, 14 weeks old arcAbeta mice and their non-transgenic littermates were immunized. The mice received four monthly vaccinations of 10 μg recombinant ankG protein in Freund's adjuvant (FA) or PBS in FA as negative controls. Serum ELISA from ankG-immunized mice showed monthly increasing titers of ankG antibody (IgG) in comparison to controls (FIG. 8a). One month after the last immunization mice were sacrificed. Immunostaining against mouse IgGs in ankG-immunized arcAbeta mice showed their presence within the β-amyloid plaques as compared to controls, suggesting that antibodies against ankG can penetrate the blood-brain-barrier reaching ankG within plaques (FIG. 8b). AnkG-immunized and control-immunized mice did not show any obvious changes in phenotype during the immunization. Immunohistochemical analysis of brain sections revealed that active ankG immunization significantly reduced the number and size of β-amyloid plaques as compared to controls (FIGS. 4a, 4b). WB analysis of brain SDS insoluble fractions showed reduced brain levels of Aβ in ankG-immunized mice as compared to controls (FIG. 4c). Expression levels of neurofilament and GAPDH were unchanged suggesting selectivity of ankG immunization for Aβ (FIG. 4 c). Quantitative ELISA of brain SDS insoluble fractions showed a significant reduction in Aβ40 and Aβ42 (FIG. 8c). In contrast to the reduced SDS-insoluble Aβ pool, the SDS-soluble Aβ42 fraction was significantly higher after ankG immunization suggesting ankG-immunization induced disaggregation of SDS-insoluble β-amyloid material with decreased insoluble Aβ40 and Aβ42 in the formic acid fraction followed by increased Aβ42 released into the soluble fraction (compare FIG. 8c versus FIG. 8g+h).

Serum ELISA from ankG-immunized arcAbeta mice did not show anti-Aβ antibodies, excluding the possibility of a general unspecific immune reaction against amyloid antigens including Aβ that could have caused Aβ clearance (FIG. 8d). As microglial cells can clear β-amyloid plaques by phagocytosis both in the presence and in the absence of antibodies against Aβ (25), it was investigated whether ankG molecules present in β-amyloid plaques were phagocytosed by microglia. Triple immunostainings for Iba1, a marker for activated microglia, APP/Aβ and ankG showed ankG immunoreactivity within activated microglia together with β-amyloid aggregates in both ankG-immunized and control-immunized arcAbeta brains (FIG. 4a). Notably, microglial density was significantly higher within the β-amyloid plaques of ankG-immunized as compared to controls (FIG. 4a, FIG. 9a). Antibody-mediated phagocytosis by microglia is a suggested mechanism for β-amyloid plaque clearance in vivo (26). Hence, the results of the experiments performed in accordance with the present invention suggest that microglia participate in the clearance of β-amyloid plaques after active ankG immunization.

To further prove the potential of active immunization on β-amyloid plaque reduction 4 months old Tg2576 mice, another mouse model for AD harbouring the human “Swedish” APP mutation (27) were actively immunized. These mice display both amyloid pathology and memory deficits. The same immunization protocol as for the arcAbeta mice was applied. The mice were sacrificed at 7 months of age, an age at which only intraneuronal Aβ is seen in this mouse model. Serum ELISA of ankG-immunized mice showed monthly increasing titres of ankG antibody (IgG) only in 4 immunized mice out of 10. This variability in the immune response against ankG can be explained by their mixed genetic background. ELISA of formic acid extracted brain SDS insoluble fractions showed reduced brain levels of Aβ42 in the ankG-immunized mice versus controls (FIGS. 9b and 8g). No significant reduction of Aβ40 was observed (FIG. 8h). Interestingly, higher levels of Aβ42 were found in the sera of these animals suggesting a higher clearance of Aβ42 versus controls (FIGS. 9c and 8i). Such an plasma spike of Aβ₄₂ was previously observed with some forms of Aβ immunotherapy that cleared soluble Aβ from the brain into the plasma via a so called peripheral “sink” mechanism (65).

### Example 4

### Antibodies Against ankG Lower Aβ Levels and Aβ-Induced Spine Loss in Hippocampal Cultures from arcAbeta Mice

To further understand the influence of anti-ankG antibodies observed in vivo it was investigated whether anti-ankG antibodies produced in arcAbeta mice after active immunization were capable of influencing Aβ levels. To this aim mouse monoclonal antibodies were produced using hybridoma cells (28). Antibodies (named mAbA) showing the strongest immunoreactivity against ankG by WB (FIG. 9d), ELISA and immunohistochemistry (data not shown) were applied to arcAbeta organotypic hippocampal slice cultures for 7 days. A reduction in Aβ40 and Aβ42 levels was observed as compared to untreated hippocampal slices using ELISA (FIG. 5a). A mouse antibody against bovine-herpes-virus-1 was used as control showing no effect on the Aβ levels. It is known that Aβ induces spine loss in hippocampal neurons (29). Therefore, the question was addressed whether anti-ankG antibodies could protect against spine loss. Sindbis virus-mediated expression of EGFP was used to allow visualization of neurons and spines (29). Hippocampal cultures from arcAbeta mice showed lower spine density as compared to non-transgenics (FIG. 5b, 5c). Treatment with mAbA abolished spine loss in arcAbeta hippocampal cultures (FIG. 5b, 5c). mAbA did not bind to Aβ40 and Aβ42 as measured by ELISA. No effect on spine density in non-transgenic cultures was observed proving a specific protective effect only on Aβ-induced spine loss (FIG. 5b, 5c).

### Example 5

### APP but not AD Interacts with ankG in the Brain

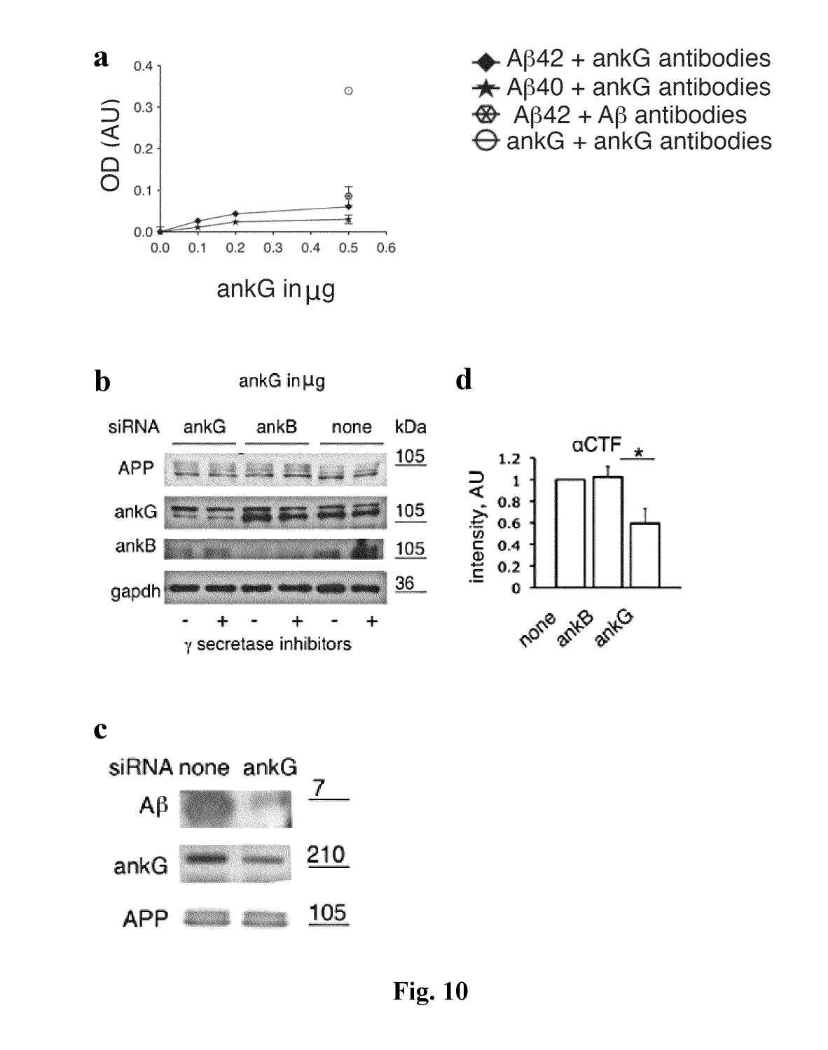

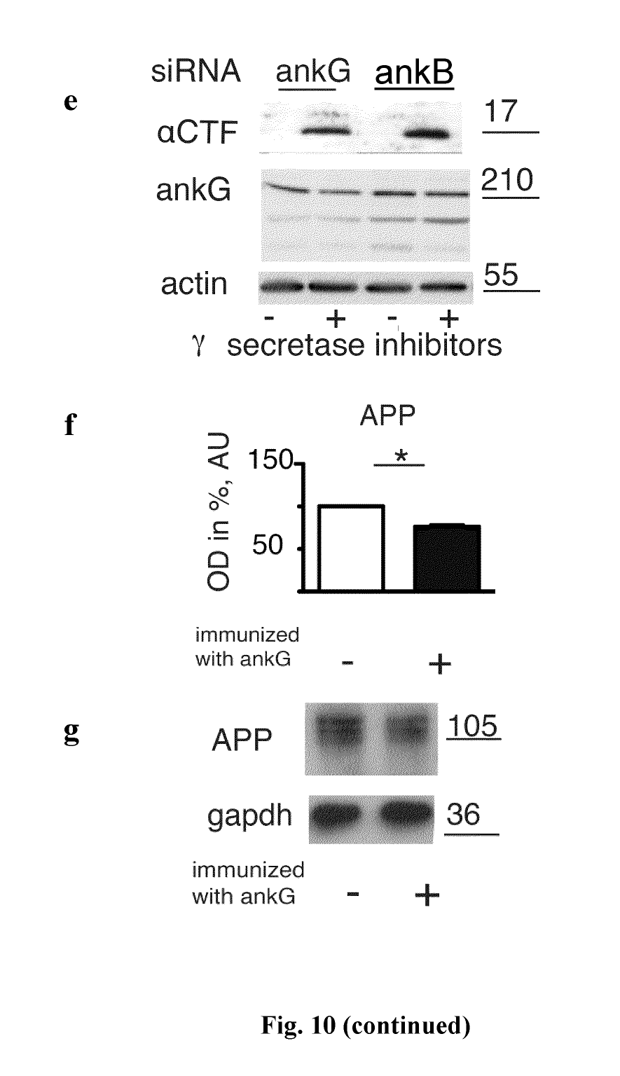

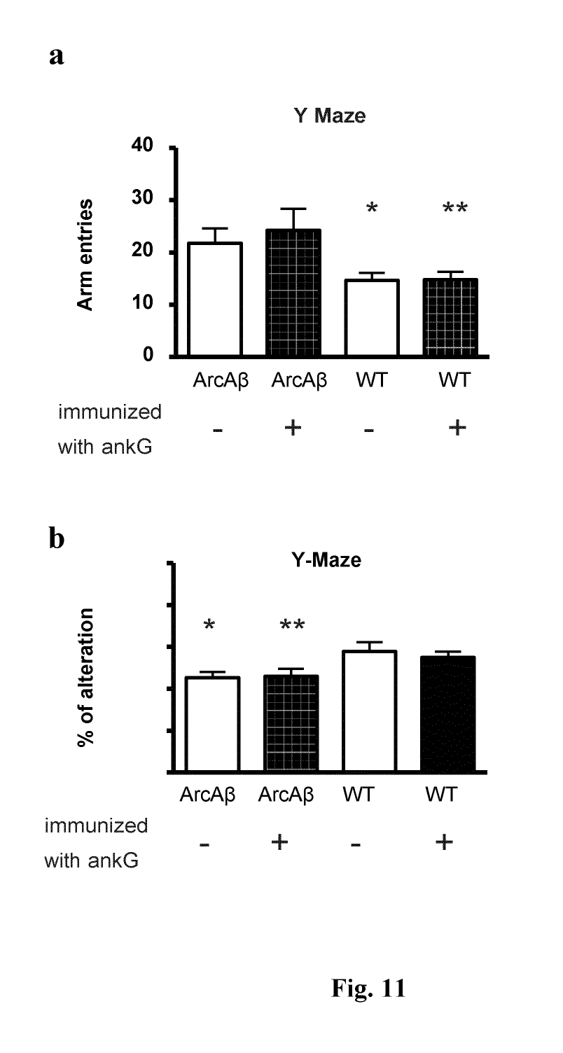

Aβ is a hydrophobic peptide and thus the presence of ankG in β-amyloid plaques could be due to a non-specific interaction with hydrophobic ankG domains. Nevertheless, ELISA performed with recombinant ankG and synthetic fibrillar Aβ (Aβ40 and Aβ42) showed no interaction between ankG and Aβ (FIG. 10a). Similarly, Aβ was absent from ankG immunoprecipitates from human and mouse brain (FIG. 6a, 6b). AnkG has been previously shown to induce clustering of adhesion molecules such as neurofascin-186 and Nr-CAM (2). APP is present on the neuronal membrane; therefore ankG could serve as its anchoring molecule. AnkG immunoprecipitation using human hippocampal homogenates showed that APP, but not Aβ, was present in the immunoprecipitated material (FIG. 6a); likewise APP co-immunoprecipitated with ankG in the reverse immunoprecipitation protocol (FIG. 6a). AnkB was not coimmunoprecipitated proving the specificity of this interaction (FIG. 6a). This specific ankG-APP interaction was also found in AD hippocampal homogenates (FIG. 6e) and in immunoprecipitates from arcAbeta and non-transgenic mice (FIG. 6b), suggesting a physiological interaction maintained also during brain β-amyloid pathology. APP-deficient mouse brains served as controls to eliminate the possibility of a non-specific interaction with IgG. Immunoprecipitation with species-specific IgG was used as negative control in all immunoprecipitation experiments (FIG. 6a). To be interacting partners two proteins have to be present in the same cellular compartment. AnkG is known to be enriched at the axonal initial segment (AIS) (2, 30) as is APP (FIG. 6c). To investigate whether the ankG-APP association is the result of a direct interaction, ELISA was performed using recombinant ankG and a synthetic peptide corresponding to the 50-amino acid cytoplasmatic fragment of APP (AICD50). It was found that ankG bound to AICD50 in a concentration-dependent manner but not to bovine serum albumin (FIG. 6d), suggesting an interaction between ankG and APP in the neuronal cytoplasm. The binding of ankG to APP and its presence in β-amyloid plaques could indicate that ankG stimulates amyloidogenic APP metabolism towards plaque formation.

### Example 6

### AnkG is Involved in APP Metabolism

To characterize the function of the interaction between ankG and APP and to determine whether ankG recruits APP to the plasma membrane, membrane proteins were biotinylated after ankG RNAi-silencing in APP-overexpressing HEK293 cells. A decrease in cell surface-biotinylated APP was observed in ankG-silenced as compared to ankB-silenced and non-silenced cells (FIG. 7a). Similar findings were found using SH-SY5Y human neuroblastoma cells and APP-citrine overexpressing HEK293 cells (FIG. 6b). Loss of ankG protein expression did not affect total APP protein levels in the cell lysates (FIG. 7b and FIG. 10b). Under physiological conditions APP is mainly cleaved within the lumenal domain by α-secretase, resulting in shedding of nearly the entire ectodomain and generation of a membrane-tethered α-C-terminal fragment (α-CTF) (31). Upon ankG silencing, decreased amounts of α-CTF were found in lysates of cells treated with γ-secretase inhibitor when compared to γ-secretase-treated ankB-silenced or non-silenced cells (FIG. 7c, FIG. 10c). The γ-secretase inhibitor was required to increase the detectable amount of α-CTF, the substrate of the γ-secretase. Similar results were observed using HEK293 cells. Similar results were also observed using SH-SY5Y cells (FIG. 10e). Brain levels of APP were decreased in ankG-immunized arcAbeta mice, with less APP in the membrane-containing SDS soluble fraction (FIG. 10f, 10g) adding in vivo evidence for the possibility that ankG contributes to the trafficking and subsequent processing of APP with resulting impact on Aβ generation. This possibility is also supported by the ELISA results of culture media of ankG-silenced Swedish APP over-expressing HeLa cells showing a reduction in Aβ40 levels as compared to controls (FIG. 7d), with levels of Aβ42 levels below detection. Moreover, ankG silencing reduced total Aβ as shown in cell lysates of HEK293 cells over-expressing Swedish APP (FIG. 10d). Together, these results indicate a role for the ankG-APP interaction in the cellular trafficking and subsequent processing of APP with a significant role in its amyloidogenic processing that ultimately results in Aβ formation and the subsequent assembly of neuropathologic aggregation products.

### Example 7

### AnkG-Immunization is not Neurotoxic and does not Interfere with Known Physiological Functions of ankG in Neurotransmission

To investigate whether ankG immunization influences known physiological functions of ankG in neurotransmission or causes any neurotoxicity, immunized and non-immunized wildtype and arcAbeta mice were subjected to Y-maze testing to measure hippocampal-dependent memory. There was no difference in behaviour in ankG-immunized wild type mice (see FIG. 11a, 11b) suggesting that ankG immunization was neither neurotoxic nor interfering with the known physiological functions of ankG in neurotransmission. Taken together with the absence of ankG antibodies in neuronal cell bodies and axons (see FIG. 8b), these findings support the idea that by ankG-reactive antibodies preferentially target extracellular ankG including ankG bound to β-amyloid plaques rather than intraneuronal ankG. Despite that no significant difference in performance between ankG-immunized and non-immunized arcAbeta mice was found, it is prudent to presume an positive effect of immunization or prolonged treatment with anti-ankG antibodies on cognitive functions of AD patients in respect of other effects of such treatment, as described above, e.g., observed protective effect of anti-ankG antibodies in hippocampal slice cultures reduction (see, FIG. 5a+b), reduced size and plaque load of β-amyloid-plaques (see, FIG. 4a+b) and higher clearance of β-amyloid (see, FIG. 4c) as discussed in further detail below.

### Discussion

AnkG is a cytoplasmic adaptor protein involved in the anchoring and assembly of voltage-gated ion channels in the axonal initial segment of neurons throughout the brain (2, 30), and genetic variations in the ankG-encoding gene ANK3 on chromosome 10q21 encoding ankG are a major risk factor for bipolar disorder (60). The results of this study establish that ankG in AD brain is abnormally expressed acquiring immunogenic properties triggering a humoral immune response with the generation of ankG-reactive IgG antibodies, and they point to a novel role of ankG in the neuropathology of AD. This is in line with the cytoskeletal pathological rearrangements known to occur in neurodegenerative disorders which affect physiological events including intracellular trafficking (13). In fact, data obtained in accordance with the present invention show that in AD, ankG—a soluble neuronal cytoplasmic protein under normal conditions—is partly relocated extracellularly to β-amyloid plaques, hence becoming an inducer of immune responses. The presence of ankG in exosomes can explain the release of this intracytoplasmic protein in the extracellular compartment and therefore its immunogenicity. This was accompanied by the presence of ankG antibodies in AD sera, wherein the frequency of ankG immunoreactive sera was higher in patients with AD as compared to aged-matched HCS. Most strikingly, cognitive decline in ankG-immunopositive AD patients was found to be lower as compared to immunonegative AD patients suggesting that ankG antibodies could be neuroprotective in humans. Serum antibodies against ankG were accompanied by extracellular accumulations of ankG in AD brains, and cortical levels of ankG were significantly higher in AD than in HCS. The partial relocalization of ankG from the neuronal cytoplasm to β-amyloid plaques in the neuropil of AD brains is possibly mediated through exosomal release as suggested by the presence of ankG in exosomes. Extracellular ankG, in particular β-amyloid plaque-associated ankG could serve as an immunogen triggering the observed humoral immune responses in AD patients as well as in aged human subjects who are known to deposit brain β-amyloid long before the onset of the clinical signs of AD. By analogy, humoral immune responses against β-amyloid are well established in AD, MC1 and non-demented aged subjects. Active immunization with ankG reduced both β-amyloid plaque load and brain concentrations of Aβ in two independent transgenic mouse models (arcAbeta and Tg2576 (“SwAPP”) mice). Reduced β-amyloid plaque load and brain concentrations of insoluble aggregates of Aβ₄₀ and Aβ₄₂ in formic acid fractions of arcAbeta mice was accompanied by increased levels of soluble Aβ₄₂ in SDS soluble fractions (see FIG. 8e), compatible with immunization-induced disaggregation of insoluble material and resulting in the release of Aβ₄₂ into the soluble pool. Aβ₄₂ has synaptotoxic activities mediated through interactions with nicotinic receptors and NMDA receptors (69) and transient increases may occur during disaggregation of fibrillar β-amyloid followed by microglial phagocytosis and clearance. It is possible that the initial disaggregation phase of β-amyloid clearance is not accompanied by immediate beneficial effects on neuronal function and behaviour, which may be expected to follow clearance of the toxic peptides and regeneration of neuronal structures (61). In experiments performed in accordance with the present invention no changes were observed in behavioural tests during the time interval of ankG immunization experiments, neither in transgenic nor in wild-type mice. This finding resembles the observation of progressive dementia in AD patients during Aβ₄₂ immunization which clearly lowered brain β-amyloid plaque load (61, 70). To address more directly the roles of ankG-reactive antibodies on the morphology of neurons subjected to Aβ-related toxicity, dendritic spine morphology in organotypic brain slices prepared from transgenic mice expressing human mutant APP was analysed. The results of these experiments showed that ankG antibodies almost completely rescued the Aβ-related spine loss in brain slices from transgenic as compared to non-transgenic wild-type littermates. This was associated with decreased levels of Aβ within the organotypic slice preparation. Because dendritic spines are known to react very sensitively to Aβ, these data suggest Aβ reduction as a potential mechanism for the beneficial effects of ankG antibodies on dendritic spines in arcAbeta mouse brains slices. Data presented hereinabove showed that microglial cells are involved in phagocytosis of ankG present within β-amyloid plaques, possibly via Fc receptor-mediated phagocytosis of IgG reacting with ankG bound to β-amyloid. While ankG immunization showed the potential to reduce and clear β-amyloid to protect neurons from Aβ-related damage, it is necessary to determine whether these potentially beneficial effects can be achieved without disrupting ankG's physiological functions in anchoring voltage-gated ion channels and adhesion molecules to the axonal cytoskeleton. This would require, however, specific neutralization of ankG in its physiological cytoplasmic localization, a compartment that is unlikely targeted by extracellular antibodies that may be internalized into luminal endosomal compartments with limited capabilities to escape into the cytoplasm. Experiments described hereinabove support the unlikeliness of such disruption of ankG's physiological functions as active immunization with ankG in mice did not lead to any obvious side effects.

Studies on passive and active immunization with Aβ proceeded rapidly to clinical trials, at which stage 6% of the patients developed meningoencephalitis (32, 33). Since then there has been a search for other immunotherapeutic candidates in AD. Recent findings showed that active immunization against the phosphorylated tau epitope in the P301L tangle mouse model reduces aggregated tau in the brain and slows progression of the tangle-related phenotype (34, 35), and alpha-synuclein immunization reduced the related pathology in transgenic mouse models (71). However, at least in respect of tau this immunization led to neurological defects caused by adverse immunogenic reactions (35). Although preliminary, active immunization with ankG in mice did not lead to any obvious side effects. AnkG immunotherapy shows promise for clearing β-amyloid. In order to achieve immunization with ankG interfering with APP trafficking and β-amyloid clearance without disrupting physiological functions of ankG one approach could be a specific immunotherapeutic subcellular targeting of ankG with no cross-reactivity against endogenously functioning ankG. Previous studies have found autoantibodies in AD patients against spectrin, glial fibrillary acidic protein, myelin basic protein and aldolase, raising the question as to whether these antibodies are neurotoxic or neuroprotective (36-40). The study performed in accordance with the present invention shows for the first time that auto-antibodies against ankG could have neuroprotective potential by lowering Aβ toxicity most likely modulating APP metabolism. In addition to its genetic association with bipolar disorder (68), ANK3, the gene encoding AnkG was found to be one of the 23 functional candidate genes associated with late onset AD (14, 41, 64). A perspective for ankG as a potential biomarker for AD is strongly supported by the present data. If immunogenicity of ankG is linked not only to AD pathology but also to disease progression, the presence of anti-ankG antibodies could serve as a biomarker. Such a possibility is addressed in the present study although but requires further investigation. Furthermore, if immunogenicity of ankG is not only linked to AD pathology but also to bipolar disorder, the presence of anti-ankG antibodies may influence the clinical course of this cerebral affection as well, also requiring further studies to address this possibility.

Bennett and co-workers showed that ankG is involved in the targeting of several membrane proteins to specialized domains of the plasma membrane (6, 42-44). AnkG is also involved in the assembly of molecules at the nodes of Ranvier and the AIS of neurons (44, 45-47). In accordance with the present invention it was be shown for the first time that ankG, by interacting directly with APP, contributes to its membrane localization and processing. The loss of surface localization of APP in the absence of ankG opens the possibility that APP is targeted by ankG to a specific micro-domain of the plasma membrane. Precise surface targeting of APP is a prerequisite for the proposed role of APP in synapse formation and stability, as well as functioning as a cell surface receptor (48). Abnormal expression of ankG as seen in AD brains taken together with the role of ankG in APP membrane targeting and Aβ production suggests an alteration of these ankG-mediated events in AD. Since it is known that altered trafficking of APP is directly linked to aberrant proteolytic processing of APP and Aβ production (49) anti-ankG antibodies could interfere with APP processing, contributing to reduction of Aβ production and therefore β-amyloid plaque formation. Previous studies describe auto-antibodies in AD patients against spectrin, glial fibrillary acidic protein, myelin basic protein and aldolase (36-40). Data presented herein of reduced cognitive decline in AD patients with positive serum titers of ankG-reactive antibodies provides evidence for a protective potential of such antibodies in AD. Should similar antibodies be present in IVIG preparations consisting of pooled IgG fractions derived from a large number of human donors, these may contribute to some of the beneficial effects of IVIG observed in clinical trials.

In accordance with the present invention it was also shown that microglial cells are involved in phagocytosis of ankG molecules present within β-amyloid plaques. It is speculated that microglia are recruited to phagocytose β-amyloid plaques by the activation of their Fc receptors through antibodies directed against brain antigens (50) which could also be the case for ankG antibodies. In conclusion, the experiments performed in accordance with the present invention demonstrate that ankG constitutes a β-amyloid-related antigen, and that AD patients generate an antibody immune response against it. This study further supports a therapeutic role for ankG antibodies in AD as underlined by the significant reduction in β-amyloid plaques via anti-ankG antibodies in vivo as well as a decrease in Aβ-induced spine loss ex vivo. The present data also show a role for ankG in cellular processing of APP.

### REFERENCES

1. Mohler, P. J., Bennett, V. 2005. Defects in ankyrin-based cellular pathways in metazoan physiology. Front. Biosci. 10: 2832-2840.
2. Dzhashiashvili, Y., Zhang, Y., Galinska, J., Lam, I., Grumet, M., Salzer J. L. 2007. Nodes of Ranvier and axon initial segments are ankyrin G-dependent domains that assemble by distinct mechanisms. J. Cell Biol. 177:857-870.
3. Tuvia, S., Garver, T. D., Bennett, V. 1997. The phosphorylation state of the FIGQY tyrosine of neurofascin determines ankyrin-binding activity and patterns of cell segregation. Proc. Natl. Acad. Sci. USA 94:12957-12962.
4. Zhou, D., Lambert, S., Malen, P. L., Carpenter, S., Boland, L. M., Bennett, V. 1998. AnkyrinG is required for clustering of voltage-gated Na channels at axon Initial segments and for normal action potential firing. J. Cell. Biol. 143:1295-1304.
5. Song, A. H., Wang, D., Chen, G., Li, Y., Luo, J., Duan, S., Poo, M. M. 2009. A selective filter for cytoplasmic transport at the axon initial segment. Cell 6:1148-1160.
6. Kizhatil, K., Baker, S. A., Arshaysky, V. Y., Bennett, V. 2009. Ankyrin-G promotes cyclic nucleotide-gated channel transport to rod photoreceptor sensory cilia. Science 323:1614-1617.
7. Fache, M-P., Moussif, A., Fernandes, F., Giraud, P., Gamido, J. J., Dargent, B. 2004. Endocytic elimination and domain-selective tethering constitute a potential mechanism of protein segregation at the axonal initial segment. J. Cell Biol. 166:571-578.
8. Shaw, A. S., Filbert, E. L. 2009. Scaffold proteins and immune-cell signalling. Nat. Rev. Immunol. 9:47-56.
9. Bennett, V., Healy, J. 2009. Membrane domains based on ankyrin and spectrin associated with cell-cell interactions. Cold Spring Harb. Perspect. Biol. 1:a003012.
10. Rasband, M. N. 2010. The axon initial segment and the maintenance of neuronal polarity. Nat. Rev. Neurosci. 11:552-562.
11. Sanchez-Ponce, D., Tapia, M., Muñoz, A., Gamido, J. J. 2008. New role of IKKα/β phosphorylated IκBα in axon outgrowth and axon initial segment development. Mol. Cell. Neurosci. 37:832-844.
12. Sethi, G., Tergaonkar, V. 2009. Potential pharmacological control of the NF-κB pathway. Trends Pharmacol. Sci. 30:313-321.
13. Bamburg, J. R., Bloom, G. S. 2009. Cytoskeletal pathologies of Alzheimer disease. Cell. Mot. Cytoskel. 66:635-649.
14. Bertram, L. et al. 2000. Evidence for genetic linkage of Alzheimer's disease to chromosome 10q. Science 290:2302-2303.
15. Wang, G., Zhang, Y., Chen, B., Cheng, J. 2003. Preliminary studies on Alzheimer's disease using cDNA microarrays. Mechan. Ageing Dev. 124:115-124.
16. Zhang, X., and Bennett, V. 1998. Restriction of 480/270-kD ankyrin G to axon proximal segments requires multiple ankyrin G-specific domains. J. Cell. Biol. 142:1571-1581.
17. Knobloch, M., Konietzko, U., Krebs, D. C., Nitsch, R. M. 2007. Intracellular Abeta and cognitive deficits precede beta-amyloid deposition in transgenic arcAbeta mice. Neurobiol. Aging 28:1297-1306.
18. Grundke-Iqbal, I., Iqbal, K., Quinlan, M., Tung, Y. C., Zaidi, M. S., Wisniewski, H. M. 1986. Microtubule-associated protein tau. A component of Alzheimer paired helical filaments. J. Biol. Chem. 261:6084-6089.
19. Aguzzi, A., Rajendran, L. 2009. The transcellular spread of cytosolic amyloids, prions, and prionoids. Neuron 64: 783-790.
20. Qazi, K. R., Gehrmann, U., Domange Jordo, E., Karlsson, M. C., Gabrielsson, S. 2009. Antigen-loaded exosomes alone induce Th1-type memory through a B-cell-dependent mechanism. Blood 113:2637-2683.
21. Mohler, P. J. 2006. Ankyrins and human disease: what the electrophysiologist should know. J. Cardiovasc. Electrophysiol. 17:1153-1159.
22. Cepok, S., et al. 2005. Identification of Epstein-Barr virus proteins as putative targets of the immune response in multiple sclerosis. J. Clin. Invest. 115:1352-1360.
23. Lalive, P. H., Menge, T., Barman, I., Cree, B. A., Genain, C. P. 2006. Identification of new serum autoantibodies in neuromyelitis optica using protein microarrays. Neurology 67:176-177.
24. Horn, S., et al. 2006. Profiling humoral autoimmune repertoire of dilated cardiomyopathy (DCM) patients and development of a disease-associated protein chip. Proteomics 6:605-613.
25. Morgan, D. 2009. The role of microglia in antibody-mediated clearance of amyloid-beta from the brain. CNS Neurol. Disord. Drug Targets 8:7-15.
26. Koenigsknecht-Talboo, J. et al. 2008. Rapid microglial response around amyloid pathology after systemic anti-Abeta antibody administration in PDAPP mice. J. Neurosci. 28:14156-14164.
27. Hsiao, K. et al. 1996. Correlative memory deficits, Abeta elevation, and amyloid plaques in transgenic mice. Science 274:99-103.
28. Koehler, G., Milstein, C. 1975. Continuous cultures of fused cells secreting antibody of predefined specificity. Nature 256:495-497.
29. Tackenberg, C., Brandt, R. 2009. Divergent pathways mediate spine alterations and cell death induced by amyloid-beta, wild-type tau, and R406W tau. J. Neurosci. 29:14439-14450.
30. Komada, M., Soriano, P. 2002. [Beta]IV-spectrin regulates sodium channel clustering through ankyrin-G at axon initial segments and nodes of Ranvier. J. Cell Biol. 156:337-348.
31. Thinakaran, G., Koo, E. H. 2008. Amyloid precursor protein trafficking, processing, and function. J. Biol. Chem. 283:29615-29619.
32. Hock, C. et al. 2003. Antibodies against beta-amyloid slow cognitive decline in Alzheimer's disease. Neuron 38:547-554.
33. Orgogozo, J. M. et al. 2003. Subacute meningoencephalitis in a subset of patients with AD after Abeta42 immunization. Neurology 61:46-54.
34. Asuni, A. A., Boutajangout, A., Quartermain, D., and Sigurdsson, E. M. 2007. Immunotherapy targeting pathological tau conformers in a tangle mouse model reduces brain pathology with associated functional improvements. J. Neurosci. 27:9115-9129.
35. Rosenmann H. et al. 2006. Tauopathy-like abnormalities and neurologic deficits in mice immunized with neuronal tau protein. Arch. Neurol. 63:1459-1467.
36. Singh, V. K., Yang, Y. Y., Singh, E. A. 1992. Immunoblot detection of antibodies to myelin basic protein in Alzheimer's disease patients. Neurosci. Lett. 147:25-28.
37. Ounanian, A., Guilbert, B., Renversez, J. C., Seigneurin, J. M., Avrameas, S. 1990.
Antibodies to viral antigens, xenoantigens, and autoantigens in Alzheimer's disease. J. Clin. Lab. Anal. 4:367-375.
38. Mor, F., Izak, M., Cohen, I. R. 2005. Identification of aldolase as a target antigen in Alzheimer's disease. J. Immunol. 175:3439-3445.
39. Mecocci, P. et al. 1995. Serum anti-GFAP and anti-S100 autoantibodies in brain aging, Alzheimer's disease and vascular dementia. J. Neuroimmunol. 57:165-170.
40. Kellner, A., Matschke, J., Bernreuther C., Moch, H., Ferrer, I., Glatzel, M. 2009. Autoantibodies against beta-amyloid are common in Alzheimer's disease and help control plaque burden. Ann. Neurol. 65:24-31.
41. Morgan, A. R. et al. 2007. Association studies of 23 positional/functional candidate genes on chromosome 10 in late-onset Alzheimer's disease. Am. J. Med. Genet. B. Neuropsychiatr. Genet. 144B:762-770.
42. Mohler, P. J. et al. 2004. Nav1.5 E1053K mutation causing Brugada syndrome blocks binding to ankyrin-G and expression of Nav1.5 on the surface of cardiomyocytes. Proc. Natl. iAcad. Sci. USA 101:17533-17538.
43. Kizhatil, K., Davis, J. Q., Davis, L., Hoffman, J., Hogan, B. L., Bennett, V. 2007. Ankyrin-G is a molecular partner of E-cadherin in epithelial cells and early embryos. J. Biol. Chem. 282:26552-26561.
44. Kizhatil, K., Yoon, W., Mohler, P. J., Davis, L. H., Hoffman, J., Bennett, V. 2007. Ankyrin-G and beta2-spectrin collaborate in biogenesis of lateral membrane of human bronchial epithelial cells. J. Biol. Chem. 282:2029-2037.
45. Jenkins, S. M., Bennett, V. 2001. Ankyrin-G coordinates assembly of the spectrin-based membrane skeleton, voltage-gated sodium channels, and L1 CAMs at Purkinje neuron initial segments. J. Cell Biol. 155:739-746.
46. Jenkins, S. M., Bennett, V. 2002. Developing nodes of Ranvier are defined by ankyrin-G clustering and are independent of paranodal axoglial adhesion. Proc. Natl. Acad. Sci. USA 99:2303-2308.
47. Pan, Z., et al. 2006. A common ankyrin-G-based mechanism retains KCNQ and NaV channels at electrically active domains of the axon. J. Neurosci. 26:2599-2613.
48. Turner, P. R., O'Connor, K., Tate, W. P., Abraham, W. C. 2003. Roles of amyloid precursor protein and its fragments in regulating neural activity, plasticity and memory. Prog. Neurobiol. 70:1-32.
49. Rajendran, L., et al. 2008. Efficient inhibition of the Alzheimer's disease beta-secretase by membrane targeting. Science 320:520-523.
50. Diamond, B., Huerta, P. T., Mina-Osorio, P., Kowal, C., and Volpe, B. T. 2009. Losing your nerves? Maybe it's the antibodies. Nat. Rev. Immunol. 9:449-456.
51. Biscaro, B., Lindvall, O., Hock, C., Ekdahl, C. T., Nitsch, R. M. 2009. Abeta immunotherapy protects morphology and survival of adult-born neurons indoubly transgenic APP/PS1 mice. J. Neurosci. 29:14108-14119.
52. Mirra, S. S. et al. 1991. The Consortium to Establish a Registry for Alzheimer's Disease (CERAD). Part II. Standardization of the neuropathologic assessment of Alzheimer's disease. Neurology 41:479-486.
53. Braak, H., Braak, E. 1991. Neuropathological stageing of Alzheimer-related changes. Acta Neuropathol. 82:239-259.
54. Goodger, Z. V., Rajendran, L., Trutzel, A., Kohli, B. M., Nitsch, R. M., Konietzko, U. 2009. Nuclear signaling by the APP intracellular domain occurs predominantly through the amyloidogenic processing pathway. J. Cell Sci. 122:3703-3714.
55. Stoppini, L., Buchs, P. A., Muller, D. 1991. A simple method for organotypic cultures of nervous tissue. J. Neurosci. Methods 37:173-182.
56. Rasband, W. S., ImageJ, U.S. National Institutes of Health, Bethesda, Md., USA, http://rsb.info.nih.gov/ij/, 1997-2009.
57. Abramoff, M. D., Magelhaes, P. J., Ram, S. J. “Image Processing with ImageJ”. Biophotonics International, volume 11, issue 7, pp. 36-42, 2004.
58. Togo, T. et al. Occurrence of T cells in the brain of Alzheimer's disease and other neurological diseases. J Neuroimmunol 124:83-92 (2002).
59. Akiyama, H. et al. Inflammation and Alzheimer's disease Neurobiol Aging 21:383-421 (2000).
60. Nitsch, R. M. and Hock, C. Targeting beta-amyloid pathology in Alzheimer's disease with Abeta immunotherapy. Neurotherapeutics. 5:415-20 (2008).
61. Serrano Pozo A. et al. Beneficial effect of human anti-Aβ active immunization (AN1792) on amyloid deposition and neurite morphology. Program No. 825.5. 2009 Neuroscience Meeting Planner (2009).
62. Bennett V. and Healy, J. Organizing the fluid membrane bilayer: diseases linked to spectrin and ankyrin. Trends Mol. Med. 14:28-36 (2008).
63. Paez-Gonzalez P. et al. Ank3-dependent SVZ niche assembly is required for the continued production of new neuron. Neuron. 71:61-75 (2011).
64. Myers A. et al. Susceptibility locus for Alzheimer's disease on chromosome 10. Science 290:2304-2305 (2000).
65. Zlokovic, B. V. eta al. Low-density lipoprotein receptor-related protein-1: a serial clearance homeostatic mechanism controlling Alzheimer's amyloid β-peptide elimination from the brain. J Neurochem. 115:1077-89.
66. Emmanouilidou, E. et al. Cell-produced alpha-synuclein is secreted in a calcium-dependent manner by exosomes and impacts neuronal survival. J. Neurosci. 30:6838-51 (2010)
67. Thinakaran, G., and Koo, E. H. Amyloid precursor protein trafficking, processing, and function. J Biol Chem 283:29615-29619 (2008).
68. Ferreira, M. A. et al. Collaborative genome-wide association analysis supports a role for ANK3 and CACNA1C in bipolar disorder. Nat. Genet. 40:1056-1058 (2008)
69. Bordji, K., Becerril-Ortega, J., Buisson, A. Synapses, NMDA receptor activity and neuronal Abeta production in Alzheimer's disease. Rev Neurosci. 22:285-94. (2011).
70. Holmes, C. et al. Long-term effects of Abeta42 immunisation in Alzheimer's disease: follow-up of a randomised, placebo-controlled phase I trial. Lancet. 372(9634):216-223 (2008).
71. Masliah, E. et al. Passive immunization reduces behavioral and neuropathological deficits in an alpha-synuclein transgenic model of Lewy body disease. PLoS One. 6:e19338 (2011).
72. Crawley, J. N., What's wrong with my mouse? Behavioral phenotyping of transgenic and knockout mice. Hoboken, N.J.: John Wiley & Sons, Inc. (2007).
73. Shahani, N. et al. Tau aggregation and progressive neuronal degeneration in the absence of changes in spine density and morphology after targeted expression of Alzheimer's disease-relevant tau constructs in organotypic hippocampal slices. J Neurosci 31:6103-6114 (2006).

1.-28. (canceled)

29. A method of reducing brain amyloid-beta protein (Aβ) pathology by lowering brain levels of Aβ comprising administering to a subject in need thereof a therapeutically effective amount of an agent selected from the group consisting of:
a) a recombinant ankG protein or a fragment, derivative or analog thereof;
b) an ankG-binding molecule;
c) an anti-ankG antibody; and,
d) a polynucleotide that reduces the expression or the cortical level of ankG protein in the brain.

30. The method of claim 29, wherein the agent is an ankG-binding molecule that is a fragment, derivative or analog of amyloid precursor protein (APP) derived from the intracytoplasmatic domain of APP.

31. The method of claim 29, wherein said agent is formulated for administration as a vaccine.

32. The method of claim 31, wherein the vaccine induces autoantibodies against ankG in the subject.

33. The method of claim 29, wherein the agent is a polynucleotide selected from the group consisting of triple helix DNA, an antisense nucleic acid, a microRNA, double stranded RNA, a ribozyme, a small interfering RNA (siRNA).

34. A method of diagnosing a neurological disorder in a subject comprising detecting in a body fluid obtained from a subject the presence of an anti-ankG autoantibody, wherein the presence of, or an elevated level of the anti-ankG autoantibody compared to the level present in a control sample from an individual that does not have a neurological disorder is indicative that the subject has or will develop a neurological disorder.

35. The method of claim 34, wherein the body fluid is cerebrospinal fluid or blood.

36. A method of in vivo imaging of ankG or ankG binding protein in the brain, comprising:
a) administering to a subject a labeled agent, wherein the agent is selected from the group consisting of:
i) a recombinant ankG protein or a fragment, derivative or analog thereof;
ii) an ankG-binding molecule;
iii) an anti-ankG antibody; and,
iv) a polynucleotide that reduces the expression or the cortical level of ankG protein in the brain.
b) scanning the subject to detect the sites of the labeled agent within the subject.


SEQUENCE LISTING (2 sequences)

SEQ ID NO: 1 - protein, 4377 aa, Homo sapiens
  ref <301>: AUTHORS: Kordeli E, Lambert S, Bennett V.
  ref <302>: TITLE: AnkyrinG. A new ankyrin gene with neural-specific isoforms localized at the axonal initial segment and node of Ranvier.
  ref <303>: JOURNAL: Journal of Biological Chemistry
  ref <304>: VOLUME: 270
  ref <305>: ISSUE: 5
  ref <306>: PAGES: 2352-2359
  ref <307>: DATE: 1995-02-03
  ref <308>: DATABASE ACCESSION NUMBER: PMID/7836469
  ref <309>: DATABASE ENTRY DATE: 1995-02-03
  ref <313>: RELEVANT RESIDUES IN SEQ ID NO: (1)..(4377)
  ref <308>: DATABASE ACCESSION NUMBER: UniProtKB/Q12955
  ref <309>: DATABASE ENTRY DATE: 2002-07-11
  ref <313>: RELEVANT RESIDUES IN SEQ ID NO: (1)..(4377)
MAHAASQLKKNRDLEINAEEEPEKKRKHRKRSRDRKKKSDANASYLRAARAGHLEKALDYIKNGVDINICNQNGLNALHLASKEGHVEVVSELLQREANVDAATKKGNTALHIASLAGQAEVVKVLVTNGANVNAQSQNGFTPLYMAAQENHLEVVKFLLDNGASQSLATEDGFTPLAVALQQGHDQVVSLLLENDTKGKVRLPALHIAARKDDTKAAALLLQNDNNADVESKSGFTPLHIAAHYGNINVATLLLNRAAAVDFTARNDITPLHVASKRGNANMVKLLLDRGAKIDAKTRDGLTPLHCGARSGHEQVVEMLLDRAAPILSKTKNGLSPLHMATQGDHLNCVQLLLQHNVPVDDVTNDYLTALHVAAHCGHYKVAKVLLDKKANPNAKALNGFTPLHIACKKNRIKVMELLLKHGASIQAVTESGLTPIHVAAFMGHVNIVSQLMHHGASPNTTNVRGETALHMAARSGQAEVVRYLVQDGAQVEAKAKDDQTPLHISARLGKADIVQQLLQQGASPNAATTSGYTPLHLSAREGHEDVAAFLLDHGASLSITTKKGFTPLHVAAKYGKLEVANLLLQKSASPDAAGKSGLTPLHVAAHYDNQKVALLLLDQGASPHAAAKNGYTPLHIAAKKNQMDIATTLLEYGADANAVTRQGIASVHLAAQEGHVDMVSLLLGRNANVNLSNKSGLTPLHLAAQEDRVNVAEVLVNQGAHVDAQTKMGYTPLHVGCHYGNIKIVNFLLQHSAKVNAKTKNGYTPLHQAAQQGHTHIINVLLQNNASPNELTVNGNTALGIARRLGYISVVDTLKIVTEETMTTTTVTEKHKMNVPETMNEVLDMSDDEVRKANAPEMLSDGEYISDVEEGEDAMTGDTDKYLGPQDLKELGDDSLPAEGYMGFSLGARSASLRSFSSDRSYTLNRSSYARDSMMIEELLVPSKEQHLTFTREFDSDSLRHYSWAADTLDNVNLVSSPIHSGFLVSFMVDARGGSMRGSRHHGMRIIIPPRKCTAPTRITCRLVKRHKLANPPPMVEGEGLASRLVEMGPAGAQFLGPVIVEIPHFGSMRGKERELIVLRSENGETWKEHQFDSKNEDLTELLNGMDEELDSPEELGKKRICRIITKDFPQYFAVVSRIKQESNQIGPEGGILSSTTVPLVQASFPEGALTKRIRVGLQAQPVPDEIVKKILGNKATFSPIVTVEPRRRKFHKPITMTIPVPPPSGEGVSNGYKGDTTPNLRLLCSITGGTSPAQWEDITGTTPLTFIKDCVSFTTNVSARFWLADCHQVLETVGLATQLYRELICVPYMAKFVVFAKMNDPVESSLRCFCMTDDKVDKTLEQQENFEEVARSKDIEVLEGKPIYVDCYGNLAPLTKGGQQLVFNFYSFKENRLPFSIKIRDTSQEPCGRLSFLKEPKTTKGLPQTAVCNLNITLPAHKKETESDQDDEIEKTDRRQSFASLALRKRYSYLTEPGMIERSTGATRSLPTTYSYKPFFSTRPYQSWTTAPITVPGPAKSGFTSLSSSSSNTPSASPLKSIWSVSTPSPIKSTLGASTTSSVKSISDVASPIRSFRTMSSPIKTVVSQSPYNIQVSSGTLARAPAVTEATPLKGLASNSTFSSRTSPVTTAGSLLERSSITMTPPASPKSNINMYSSSLPFKSIITSAAPLISSPLKSVVSPVKSAVDVISSAKITMASSLSSPVKQMPGHAEVALVNGSISPLKYPSSSTLINGCKATATLQEKISSATNSVSSVVSAATDTVEKVFSTTTAMPFSPLRSYVSAAPSAFQSLRTPSASALYTSLGSSISATTSSVTSSIITVPVYSVVNVLPEPALKKLPDSNSFTKSAAALLSPIKTLTTETHPQPHFSRTSSPVKSSLFLAPSALKLSTPSSLSSSQEILKDVAEMKEDLMRMTAILQTDVPEEKPFQPELPKEGRIDDEEPFKIVEKVKEDLVKVSEILKKDVCVDNKGSPKSPKSDKGHSPEDDWIEFSSEEIREARQQAAASQSPSLPERVQVKAKAASEKDYNLTKVIDYLTNDIGSSSLTNLKYKFEDAKKDGEERQKRVLKPAIALQEHKLKMPPASMRTSTSEKELCKMADSFFGTDTILESPDDFSQHDQDKSPLSDSGFETRSEKTPSAPQSAESTGPKPLFHEVPIPPVITETRTEVVHVIRSYDPSAGDVPQTQPEEPVSPKPSPTFMELEPKPTTSSIKEKVKAFQMKASSEEDDHNRVLSKGMRVKEETHITTTTRMVYHSPPGGEGASERIEETMSVHDIMKAFQSGRDPSKELAGLFEHKSAVSPDVHKSAAETSAQHAEKDNQMKPKLERIIEVHIEKGNQAEPTEVIIRETKKHPEKEMYVYQKDLSRGDINLKDFLPEKHDAFPCSEEQGQQEEEELTAEESLPSYLESSRVNTPVSQEEDSRPSSAQLISDDSYKTLKLLSQHSIEYHDDELSELRGESYRFAEKMLLSEKLDVSHSDTEESVTDHAGPPSSELQGSDKRSREKIATAPKKEILSKIYKDVSENGVGKVSKDEHFDKVTVLHYSGNVSSPKHAMWMRFTEDRLDRGREKLIYEDRVDRTVKEAEEKLTEVSQFFRDKTEKLNDELQSPEKKARPKNGKEYSSQSPTSSSPEKVLLTELLASNDEWVKARQHGPDGQGFPKAEEKAPSLPSSPEKMVLSQQTEDSKSTVEAKGSISQSKAPDGPQSGFQLKQSKLSSIRLKFEQGTHAKSKDMSQEDRKSDGQSRIPVKKIQESKLPVYQVFAREKQQKAIDLPDESVSVQKDFMVLKTKDEHAQSNEIVVNDSGSDNVKKQRTEMSSKAMPDSFSEQQAKDLACHITSDLATRGPWDKKVFRTWESSGATNNKSQKEKLSHVLVHDVRENHIGHPESKSVDQKNEFMSVTERERKLLTNGSLSEIKEMTVKSPSKKVLYREYVVKEGDHPGGLLDQPSRRSESSAVSHIPVRVADERRMLSSNIPDGFCEQSAFPKHELSQKLSQSSMSKETVETQHFNSIEDEKVTYSEISKVSKHQSYVGLCPPLEETETSPTKSPDSLEFSPGKESPSSDVFDHSPIDGLEKLAPLAQTEGGKEIKTLPVYVSFVQVGKQYEKEIQQGGVKKIISQECKTVQETRGTFYTTRQQKQPPSPQGSPEDDTLEQVSFLDSSGKSPLTPETPSSEEVSYEFTSKTPDSLIAYIPGKPSPIPEVSEESEEEEQAKSTSLKQTTVEETAVEREMPNDVSKDSNQRPKNNRVAYIEFPPPPPLDADQIESDKKHHYLPEKEVDMIEVNLQDEHDKYQLAEPVIRVQPPSPVPPGADVSDSSDDESIYQPVPVKKYTFKLKEVDDEQKEKPKASAEKASNQKELESNGSGKDNEFGLGLDSPQNEIAQNGNNDQSITECSIATTAEFSHDTDATEIDSLDGYDLQDEDDGLTESDSKLPIQAMEIKKDIWNTEGILKPADRSFSQSKLEVIEEEGKVGPDEDKPPSKSSSSEKTPDKTDQKSGAQFFTLEGRHPDRSVFPDTYFSYKVDEEFATPFKTVATKGLDFDPWSNNRGDDEVFDSKSREDETKPFGLAVEDRSPATTPDTTPARTPTDESTPTSEPNPFPFHEGKMFEMTRSGAIDMSKRDFVEERLQFFQIGEHTSEGKSGDQGEGDKSMVTATPQPQSGDTTVETNLERNVETPTVEPNPSIPTSGECQEGTSSSGSLEKSAAATNTSKVDPKLRTPIKMGISASTMTMKKEGPGEITDKIEAVMTSCQGLENETITMISNTANSQMGVRPHEKHDFQKDNFNNNNNLDSSTIQTDNIMSNIVLTEHSAPTCTTEKDNPVKVSSGKKTGVLQGHCVRDKQKVLGEQQKTKELIGIRQKSKLPIKATSPKDTFPPNHMSNTKASKMKQVSQSEKTKALTTSSCVDVKSRIPVKNTHRDNIIAVRKACATQKQGQPEKGKAKQLPSKLPVKVRSTCVTTTTTTATTTTTTTTTTTTSCTVKVRKSQLKEVCKHSIEYFKGISGETLKLVDRLSEEEKKMQSELSDEEESTSRNTSLSETSRGGQPSVTTKSARDKKTEAAPLKSKSEKAGSEKRSSRRTGPQSPCERTDIRMAIVADHLGLSWTELARELNFSVDEINQIRVENPNSLISQSFMLLKKWVTRDGKNATTDALTSVLTKINRIDIVTLLEGPIFDYGNISGTRSFADENNVFHDPVDGWQNETSSGNLESCAQARRVTGGLLDRLDDSPDQCRDSITSYLKGEAGKFEANGSHTEITPEAKTKSYFPESQNDVGKQSTKETLKPKIHGSGHVEEPASPLAAYQKSLEETSKLIIEETKPCVPVSMKKMSRTSPADGKPRLSLHEEEGSSGSEQKQGEGFKVKTKKEIRHVEKKSHS

SEQ ID NO: 2 - protein, 1001 aa, Homo sapiens
  ref <301>: AUTHORS: Kapfhamer D, Miller DE, Lambert S, Bennett V, Glover TW, Burmeister M.
  ref <302>: TITLE: Chromosomal localization of the ankyrinG gene (ANK3/Ank3) to human 10q21 and mouse 10.
  ref <303>: JOURNAL: Genomics
  ref <304>: VOLUME: 27
  ref <305>: ISSUE: 1
  ref <306>: PAGES: 189-91
  ref <307>: DATE: 1995-05-01
  ref <308>: DATABASE ACCESSION NUMBER: PMID/7665168
  ref <309>: DATABASE ENTRY DATE: 1995-10-12
  ref <313>: RELEVANT RESIDUES IN SEQ ID NO: (1)..(1001)
  ref <301>: AUTHORS: Lee MT, Chen CH, Lee CS, Chen CC, Chong MY, Ouyang WC, Chiu NY, Chuo LJ, Chen CY, Tan HK, Lane HY, Chang TJ, Lin CH, Jou SH, Hou YM, Feng J, Lai TJ, Tung CL, Chen TJ, Chang CJ, Lung FW, Chen CK, Shiah IS, Liu CY, Teng PR, Chen KH, Shen LJ, Cheng CS, Chang TP, Li CF, Chou CH, Chen CY, Wang KH, Fann CS, Wu JY, Chen YT, Cheng AT.
  ref <302>: TITLE: Genome-wide association study of bipolar I disorder in the Han Chinese population.
  ref <303>: JOURNAL: Molecular Psychiatry
  ref <304>: VOLUME: 00
  ref <306>: PAGES: 00
  ref <307>: DATE: 2010-04-13
  ref <308>: DATABASE ACCESSION NUMBER: PMID/20386566
  ref <309>: DATABASE ENTRY DATE: 2010-04-13
  ref <301>: AUTHORS: Williams HJ, Craddock N, Russo G, Hamshere ML, Moskvina V, Dwyer S, Smith RL, Green E, Grozeva D, Holmans P, Owen MJ, O′Donovan MC
  ref <302>: TITLE: Most genome-wide significant susceptibility loci for schizophrenia and bipolar disorder reported to date cross-traditional diagnostic boundaries.
  ref <303>: JOURNAL: Human Molecular Genetics
  ref <304>: VOLUME: 20
  ref <305>: ISSUE: 2
  ref <306>: PAGES: 387-391
  ref <307>: DATE: 2011-01-15
  ref <308>: DATABASE ACCESSION NUMBER: PMID/21037240
  ref <309>: DATABASE ENTRY DATE: 2010-12-23
  ref <313>: RELEVANT RESIDUES IN SEQ ID NO: (1)..(1001)
  ref <308>: DATABASE ACCESSION NUMBER: UniProtKB/B1AQT2
  ref <309>: DATABASE ENTRY DATE: 2008-04-08
  ref <313>: RELEVANT RESIDUES IN SEQ ID NO: (1)..(1001)
MALPQSEDAMTGDTDKYLGPQDLKELGDDSLPAEGYMGFSLGARSASLRSFSSDRSYTLNRSSYARDSMMIEELLVPSKEQHLTFTREFDSDSLRHYSWAADTLDNVNLVSSPIHSGFLVSFMVDARGGSMRGSRHHGMRIIIPPRKCTAPTRITCRLVKRHKLANPPPMVEGEGLASRLVEMGPAGAQFLGPVIVEIPHFGSMRGKERELIVLRSENGETWKEHQFDSKNEDLTELLNGMDEELDSPEELGKKRICRIITKDFPQYFAVVSRIKQESNQIGPEGGILSSTTVPLVQASFPEGALTKRIRVGLQAQPVPDEIVKKILGNKATFSPIVTVEPRRRKFHKPITMTIPVPPPSGEGVSNGYKGDTTPNLRLLCSITGGTSPAQWEDITGTTPLTFIKDCVSFTTNVSARFWLADCHQVLETVGLATQLYRELICVPYMAKFVVFAKMNDPVESSLRCFCMTDDKVDKTLEQQENFEEVARSKDIEVLEGKPIYVDCYGNLAPLTKGGQQLVFNFYSFKENRLPFSIKIRDTSQEPCGRLSFLKEPKTTKGLPQTAVCNLNITLPAHKKIEKTDRRQSFASLALRKRYSYLTEPGMSPQSPCERTDIRMAIVADHLGLSWTELARELNFSVDEINQIRVENPNSLISQSFMLLKKWVTRDGKNATTDALTSVLTKINRIDIVTLLEGPIFDYGNISGTRSFADENNVFHDPVDGYPSLQVELETPTGLHYTPPTPFQQDDYFSDISSIESPLRTPSRLSDGLVPSQGNIEHSADGPPVVTAEDASLEDSKLEDSVPLTEMPEAVDVDESQLENVCLSWQNETSSGNLESCAQARRVTGGLLDRLDDSPDQCRDSITSYLKGEAGKFEANGSHTEITPEAKTKSYFPESQNDVGKQSTKETLKPKIHGSGHVEEPASPLAAYQKSLEETSKLIIEETKPCVPVSMKKMSRTSPADGKPRLSLHEEEGSSGSEQKQGEGFKVKTKKEIRHVEKKSHS